(19) United States
(12) Patent Application Publication
(10) Pub. No.: US 2026/0091197 A1
(43) Pub. Date: Apr. 2, 2026

(54) MULTI-LUMEN CATHETERS AND RELATED METHODS

(71) Applicant: Cerinfuse Technologies, Inc., Tampa, FL (US)

(72) Inventor: Michael A. Vogelbaum, Tampa, FL (US)

(73) Assignee: Cerinfuse Technologies, Inc., Tampa, FL (US)

(21) Appl. No.: 19/345,302

(22) Filed: Sep. 30, 2025

Related U.S. Application Data
(60) Provisional application No. 63/701,886, filed on Oct. 1, 2024.

(51) Int. Cl. A61M 25/00; A61M 5/14; A61M 39/10; A61M 39/20

(52) U.S. Cl. CPC A61M 25/0026; A61M 5/1413; A61M 39/105; A61M 39/20; A61M 2039/1077; A61M 2210/0693

(57) ABSTRACT

This disclosure generally relates to multi-lumen catheters, ports, and adapters for delivering fluid to a tissue or cavity in a subject and methods thereof. The multi-lumen catheters disclosed herein comprise a catheter comprising a guide lumen configured to receive a stylet (e.g., for placement of the catheter within a cavity/tissue) or a stylet port of the adapter (e.g., to anchor the catheter to the adapter); and a fluid lumen configured to receive a fluid port of the adapter (e.g., to receive a fluid via the fluid port into the fluid lumen). In some embodiments, the multi-lumen catheter is size-adjustable. Other aspects of the disclosure relate to systems comprising multi-lumen catheters in fluidic communication with an adapter in fluidic communication with a reservoir. Additional aspects of the disclosure relate to methods of inserting the described catheters, ports, adapters, and/or systems into a tissue or cavity of a subject (e.g., brain tissue).

### RELATED APPLICATIONS

This application claims priority under 35 U.S.C. § 119(e) to U.S. Provisional Patent Application No. 63/701,886, filed Oct. 1, 2024, and entitled “Multi-Lumen Catheters and Related Methods,” which is incorporated herein by reference in its entirety for all purposes.

### FIELD

This disclosure generally relates to multi-lumen catheters, adapters, ports, and reservoirs, as well as the devices and systems comprising the same, for delivering fluid to a tissue or cavity in a subject and methods thereof.

### BACKGROUND

Several medical conditions, including treatment of solid tumors, require chronic administration of one or more therapeutic agents. For example, following surgical excision of a solid tumor, it is advantageous to provide a therapeutic fluid via a localized catheter to treat residual cancer cells. Providing localized therapies to the tissue can often limit the extent of debulking of the solid tumor from the diseased tissue, which in turn can help to preserve organ function. This can be critical when the neoplastic tissue is, for example, in the brain.

There are several existing systems and methods for providing interstitial treatment after a surgical excision. One method includes inserting drug-infused wafers into a surgically created cavity. However, while these wafers release the drug into the immediate vicinity of the cavity, they lack the ability to target the drug to desired location within a tissue.

An alternative approach is to use convention enhanced delivery (CED) which uses fluid pressure to push the drug into the interstitial spaces of the diseased tissue. CED based delivery methods typically require multiple catheters that are individually implanted directly into the tissue surrounding a cavity, for example, created by surgical excision of a solid tumor. The catheters are usually inserted from multiple points of origin outside the cavity, and a pump forces the drug through the catheters into the tissue in which the catheter is inserted. The open ends of the catheters are precisely positioned to ensure that the drug is delivered to the target tissue rather than the surrounding healthy tissue.

The target tissue, however, can be extensive and irregularly shaped. Current systems and methods, such as those described in International Patent Publication No. WO 2008/020967 to Matsuura et al., account for extensive and irregularly shaped regions of interest, but are complicated and difficult to manufacture, and do not prevent backflow of the drug, thus leading to sub-therapeutic levels of drug poorly distributed throughout the target tissue. Thus, a need exists for devices and/or methods that reduce back flow and leakage of the drug from the device (e.g., during CED) and that promote homogenous distribution of drug at therapeutic levels to irregularly shaped target tissues.

### SUMMARY

This disclosure generally relates to multi-lumen catheters, adapters, ports, and reservoirs, as well as the devices and systems comprising the same, for delivering fluid to a tissue or cavity in a subject and methods thereof. The subject matter of the present disclosure involves, in some cases, interrelated products, alternative solutions to a particular problem, and/or a plurality of different uses of one or more systems and/or articles.

Aspects of the disclosure relate to a system. In some embodiments, the system comprises a multi-lumen catheter, the catheter comprising a fluidic lumen, a guide lumen adjacent the fluidic lumen, and a tube. In some embodiments, the tube comprises a central elongate passageway. In some embodiments, the tube is sized and configured to be reversibly positioned within at least a portion of the fluidic lumen such that the tube extends beyond a distal end of the multi-lumen catheter. The system, in some embodiments, further comprises a fitted adapter, and a reservoir. The reservoir comprises a fluid adapter port, according to some embodiments, comprising one or more openings. In some embodiments, each opening corresponds to one or more features of the fitted adapter such that the fitted adapter connects to the fluid adapter port thereby positioning the reservoir in fluidic communication with the fluidic lumen of the multi-lumen catheter.

In some embodiments, a system is configured for the administration of a fluid to a subject. In some embodiments, the system comprises a catheter comprising a first lumen and a second lumen, the first lumen having a first diameter and the second lumen having a second diameter. The system further comprises a fluidic reservoir, the fluidic reservoir comprising a fluid adapter port having a first opening having a first cross-sectional shape, according to some embodiments. Additionally, in some embodiments, the system further comprises a fitted adapter comprising a first plug, a second plug, and a third plug. In some embodiments, the first plug has a second cross-sectional shape corresponding to the first cross-sectional shape. In some embodiments, the second plug sized and adapted to operably connect to the first lumen, wherein the first plug and the second plug are in fluidic communication. In some embodiments, the third plug is sized and adapted to operably connect to the second lumen, wherein the third plug is not in fluidic communication with the first plug. In some embodiments, the second plug has a first surface, and the third plug has a second surface, wherein a shape of a planar surface of the first surface is different than a shape of a planar surface of the second surface.

Other aspects of the disclosure relate to a device. In some embodiments, the device comprises a size-adjustable catheter comprising a fluidic lumen and a guide lumen. In some embodiments, the size-adjustable catheter further comprising a tube, the tube comprising a central elongate passageway. In some embodiments, the tube is sized and configured to be reversibly positioned within at least a portion of the fluidic lumen such that the tube extends beyond a distal end of the multi-lumen catheter. Additionally, in some embodiments, the device further comprises an adapter comprising a fluid port configured to be positioned within a proximal end of the fluidic lumen of the catheter and a stylet port configured to be positioned within a proximal end of the guide lumen of the multi-lumen catheter.

Additional aspects of the disclosure relate to an adapter. In some embodiments, the adapter is an adapter for a medical device, although this is not a requirement of the adapter. In some embodiments, the adapter comprises a connector port positioned at a first end of the adapter, a fluid port positioned at a second end of the adapter and in fluidic communication with the connector port, and a stylet port, proximate the fluid port. In some embodiments, the stylet port comprises a first surface and a second surface, the first surface and second surface having different curvatures. In some embodiments, at least a portion of the first surface extends beyond a distal portion of the fluid port.

Other aspects of the disclosure relate to methods. In some embodiments, the methods relate to inserting a catheter in a tissue of a subject. In some embodiments, the method comprises loading a stylet into a guide lumen at a proximal end of the multi-lumen catheter, the catheter further comprising a fluidic lumen and a tube comprising a central elongate passageway configured to be positioned within at least a portion of the fluidic lumen and to extend beyond a distal end of the multi-lumen catheter. In some embodiments, the methods comprise using the stylet to insert and advance the catheter to a target location within the tissue of the subject. In some embodiments, the methods comprise removing the stylet from the guide lumen of the catheter and cutting the catheter to a final length, and inserting an adapter comprising a connector port, a fluid port and a stylet port. In some embodiments, the fluid port is connected to a proximal end of the fluidic lumen of the catheter and the stylet port is connected to the proximal end of the guide lumen of the multi-lumen catheter.

Other methods relate to inserting a catheter into a cavity of a subject. In some embodiments, the methods comprise loading a stylet into a guide lumen at a proximal end of the multi-lumen catheter, the catheter further comprising a fluidic lumen and a tube comprising a central elongate passageway configured to be positioned within at least a portion of the fluidic lumen and to extend beyond a distal end of the multi-lumen catheter. In some embodiments, the methods comprise using the stylet to insert and advance the catheter into the cavity of the subject, removing the stylet from the guide lumen of the catheter and cutting the catheter to a final length, and inserting a stylet port of an adapter into the proximal end of the guide lumen of the catheter and a fluid port of the adapter into the proximal end of the fluidic lumen of the multi-lumen catheter, the adapter further comprising a connector port.

Other methods still relate to inserting a brain port comprising a reservoir in fluidic communication with a multi-lumen catheter in a brain of a subject. In some embodiments, the multi-lumen catheter comprises a fluidic lumen and a guide lumen and a tube comprising a central elongate passageway configured to be positioned within at least a portion of the fluidic lumen and to extend beyond at a distal end of the multi-lumen catheter. In some embodiments, the method comprises inserting a connector port of an adapter into a fluid adapter port of the reservoir, the adapter further comprising an fluid port and a stylet port. In some embodiments, the methods further comprise inserting the reservoir into a subgaleal space, and loading a stylet at a proximal end of the guide lumen of the multi-lumen catheter and advancing the distal end of the multi-lumen catheter through the brain until a target location is reached. In some embodiments, the methods further comprise removing the stylet from the proximal end of the guide lumen of the multi-lumen catheter and cutting the multi-lumen catheter to a final length, and connecting the stylet port of the adapter to the proximal end of the guide lumen of the multi-lumen catheter and the fluid port of the adapter to the proximal end of the fluidic lumen of the multi-lumen catheter.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In one aspect, the present disclosure encompasses methods of making one or more of the embodiments described herein, for example, a multi-lumen catheter. In another aspect, the present disclosure encompasses methods of using one or more of the embodiments described herein, for example, a multi-lumen catheter.

Other advantages and novel features of the present disclosure will become apparent from the following detailed description of various non-limiting embodiments of the disclosure when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1E show a series of illustrations of a multi-lumen catheter, according to some embodiments of the present invention. FIG. 1A illustrates a front-view of the multi-lumen catheter, FIG. 1B shows a top down view of the multi-lumen catheter, according to some embodiments. FIG. 1C shows graduation markings present on the side of the multi-lumen catheter, according to some embodiments.

FIGS. 2A-2C show a series of illustration of an adapter, according to some embodiments of the present invention. FIG. 2A shows an angled front view of the adapter, according to some embodiments. FIG. 2B shows a cross-sectional side view of the adapter, according to some embodiments. FIG. 2C shows a front view of the adapter, according to some embodiments.

FIGS. 3A and 3B show a series of illustrations of a reservoir, according to some embodiments of the present invention. FIG. 3A shows a top-down view of a reservoir comprising a base, a rigid inner plate, and a top dome. FIG. 3B shows a cross-sectional side view of the reservoir.

FIG. 4 shows an exemplary device, according to some embodiments of the present invention. The device, according to some embodiments, comprises the adapter as shown in FIG. 2 and the catheter as described in FIG. 1. In some embodiments, at least a part of the adapter is in fluidic communication with at least a part of the multi-lumen catheter.

FIG. 5 shows an illustration of a system, according to some embodiments of the present invention. The system, according to some embodiments, comprises the reservoir as shown in FIG. 3, the adapter as shown in FIG. 2, and/or the catheter as described in FIG. 1. In some embodiments, the catheter is flexible and is configured to be positioned between −90 degrees and 90 degrees relative to a first axis that runs parallel to a fluid port of a fluid adapter.

FIGS. 6A and 6B shows another exemplary adapter, according to some embodiments of the present invention. FIG. 6A shows a threaded connector port of the adapter, according to some embodiments. FIG. 6B shows a cross-sectional side view of the adapter, according to some embodiments. The fluid path extends from the fluid port to the connect port along the dotted line, in some embodiments.

### DETAILED DESCRIPTION

This disclosure generally relates to multi-lumen catheters, adapters, and ports for delivering fluid to a tissue or cavity in a subject and methods thereof. The multi-lumen catheters disclosed herein comprise a catheter comprising a guide lumen configured to receive a stylet (e.g., for placement of the catheter within a cavity/tissue) or a stylet port of the adapter (e.g., to anchor the catheter to the adapter); and a fluid lumen configured to receive a fluid port of the adapter (e.g., to receive a fluid via the fluid port into the fluid lumen). In some embodiments, the multi-lumen catheter is size-adjustable. Other aspects of the disclosure relate to systems comprising multi-lumen catheters in fluidic communication with an adapter in fluidic communication with a reservoir. Additional aspects of the disclosure relate to methods of inserting the described catheters, adapters, ports, and/or systems into a tissue or cavity (e.g., brain tissue) of a subject.

A “subject” as used herein refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the articles, devices and/or systems described herein.

The present disclosure generally describes articles such as catheters, adapters, reservoirs, ports, as well as devices and systems comprising the same, that may address one or more problems and/or deficiencies in the art, as briefly discussed above. In particular, the catheters, adapters, reservoirs, ports, and devices and systems comprising the same, as disclosed herein, use simple and easily manufacturable components to reduce and/or eliminate back flow and leakage of a therapeutic fluid from a catheter during delivery, e.g., during CED. This is accomplished, for example, by strategically placing a plug at a distal portion of the multi-lumen catheter and by ensuring multiple points of attachment are used to anchor the multi-lumen catheter to a fluid reservoir. The skilled artisan will understand, that other methods of occluding the distal portion of the multi-lumen catheter are possible and that any such method known to the skilled artisan may be used herein. Anchoring may be achieved via friction fit (e.g., engaging a barbed end of an adapter with an inner cross-sectional dimension of a lumen of the multi-lumen catheter), pressure fit, sutures (e.g., tying a knot around a multi-lumen catheter in contact with the end of an adapter), or a combination thereof. In some embodiments, such configurations ensure that fluid flow is restricted to a single fluid filled lumen of the multi-lumen catheter (e.g., prevents backflow through the guide lumen), and in the case where the catheter is connected to a port (e.g., a brain port), ensures that the catheter does not disengage from the reservoir (e.g., multiple anchor points prevent fluid from leaking out of the reservoir). Without wishing to be bound by any particular theory, it is believed that such configurations may improve both the distribution and concentration of drugs delivered to the target tissue.

The catheters, adapters, ports, and reservoirs of the instant disclosure, as well as the devices and systems comprising the same, may be tailored to fit within any body tissue (e.g., brain, kidney, heart, liver, stomach, intestine, bladder, spleen, etc.). This is possible because the catheters disclosed herein are comprised of polymers (e.g., silicone elastomers) that can be easily cut with scissors or a surgical blade following placement at the target tissue. For example, a proximal portion of a multi-lumen catheter positioned at a first point in the body (e.g., extending from a burr hole in a skull) may be connected to a reservoir positioned at a second point in the body (e.g., within the brain tissue) by cutting the proximal portion to the appropriate length (e.g., using a pair of scissors or surgical blade), discarding the cut material, and connecting the cut end of the proximal portion of the multi-lumen catheter to the adapter of the reservoir. This is useful, for example, for minimizing dead space in the multi-lumen catheter and for preventing kinks and other obstructions that may arise when using catheters of a fixed length.

Additionally, the catheters, adapters, ports, and reservoirs of the instant disclosure, as well as the devices and systems comprising the same, may be tailored to fit within any sized cavity (e.g., surgically created cavity). This is possible because the catheters disclosed herein are comprised of polymers (e.g., silicone elastomers) that can be easily cut with scissors or a surgical blade following placement at the target tissue. For example, a proximal portion of a multi-lumen catheter positioned at a first point in the body (e.g., extending from a burr hole in the skull) may be connected to a reservoir positioned at a second point in the body (e.g., with a subgaleal cavity) by cutting the proximal portion to the appropriate length (e.g., using a pair of scissors or surgical blade), discarding the cut material, and connecting the cut end of the proximal portion of the multi-lumen catheter to the adapter of the reservoir. This is useful, for example, for minimizing dead space in the multi-lumen catheter and for preventing kinks and other obstructions that may arise when using catheters of a fixed length.

In some embodiments, the multi-lumen catheter comprises a first lumen and a second lumen, the first lumen having a first diameter and the second lumen having a second diameter, as described in more detail below. In some embodiments, the multi-lumen catheter comprises a first (e.g., fluidic) lumen, a second (e.g., guide) lumen adjacent the fluidic lumen, and a tube, the tube comprising a central elongate passageway. In some embodiments, the tube is sized and configured to be reversibly positioned within at least a portion of the fluidic lumen such that the tube extends beyond a distal end of the multi-lumen catheter.

In some embodiments, a fitted adapter is provided. In some embodiments, the fitted adapter comprises two or more plugs (e.g., a first plug, a second plug, and a third plug).

In some embodiments, a reservoir comprising a fluid adapter port is provided. In some embodiments, the fluid adapter port comprises one or more openings, each opening corresponding to one or more features of the fitted adapter such that the fitted adapter connects to the fluid adapter port thereby positioning the reservoir in fluidic communication with the fluidic lumen of the multi-lumen catheter.

In some embodiments, the fluidic reservoir comprising the fluid adapter port has a first opening having a first cross-sectional shape. In some embodiments, the first plug having a second cross-sectional shape corresponding to the first cross-sectional shape, the second plug is sized and adapted to operably connect to the first lumen, wherein the first plug and the second plug are in fluidic communication, and the third plug is sized and adapted to operably connect to the second lumen, wherein the third plug is not in fluidic communication with the first plug, the second plug having a first surface, the third plug having a second surface, wherein a shape of a planar surface of the first surface is different than a shape of a planar surface of the second surface.

In some embodiments, as described in more detail below, the catheter is size-adjustable. In some embodiments, the catheter further comprises a tube, the tube comprising a central elongate passageway, wherein the tube is sized and configured to be reversibly positioned within at least a portion of the fluidic lumen such that the tube extends beyond a distal end of the multi-lumen catheter.

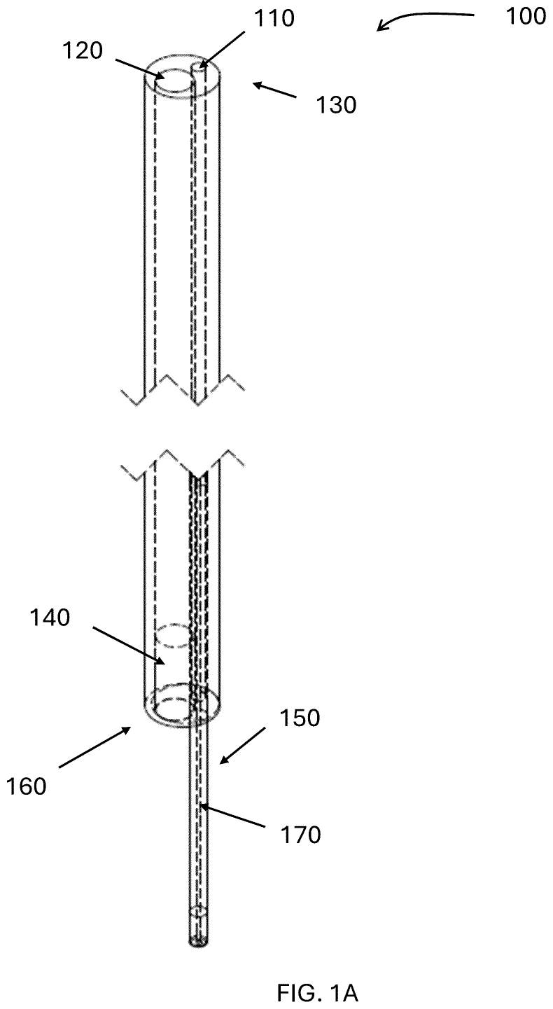

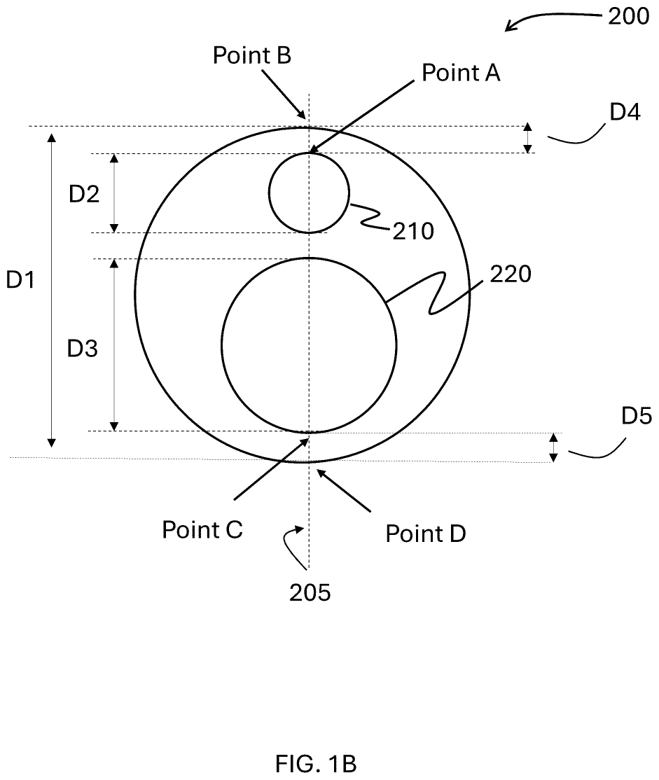

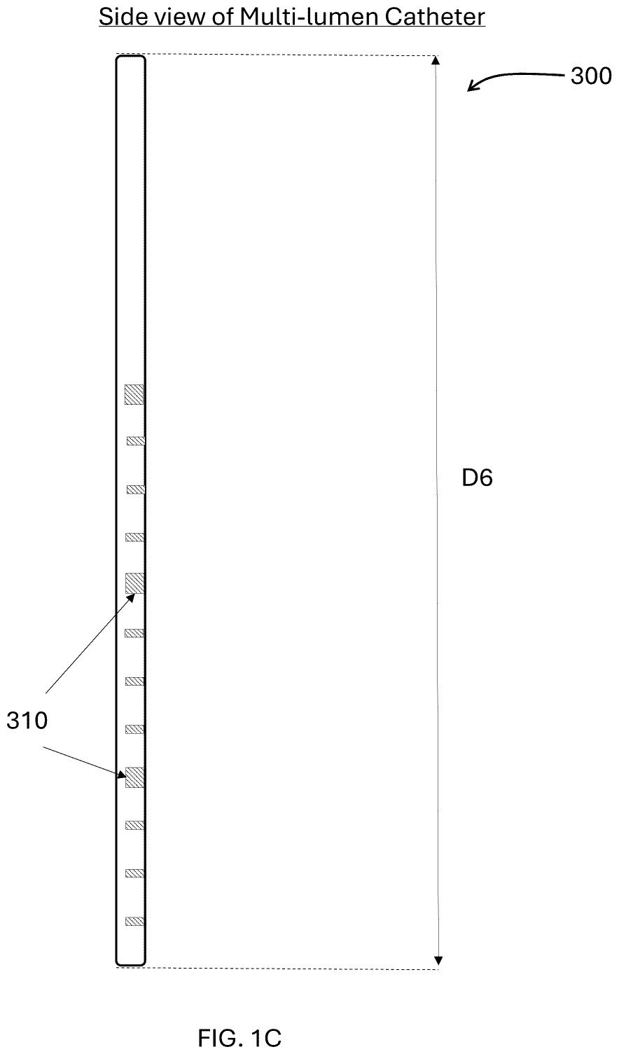

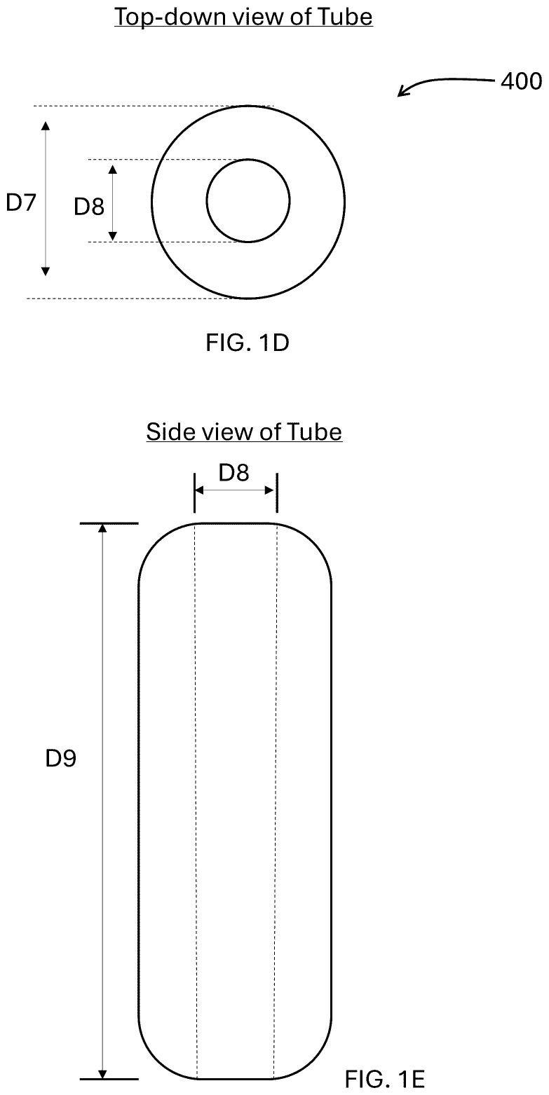

FIG. 1A shows exemplary multi-lumen catheter 100, according to some embodiments of the present invention. In some embodiments, multi-lumen catheter 100 comprises a tubular structure comprising a fluidic lumen 110 and a guide lumen 120, adjacent fluidic lumen 110, that run the entire distance of the tubular structure. In some embodiments, multi-lumen catheter 100 further comprises tube 150 sized and configured to be reversibly positioned within at least a portion of fluidic lumen 110 such that it extends beyond a distal end 160 of the multi-lumen catheter 100. Additionally, according to some embodiments, an adhesive plug 140 is positioned at a distal end 160 of the guide lumen 120 (e.g., the distal end of the guide lumen is sealed).

FIG. 1B shows a top-down view of an exemplary multi-lumen catheter 200, according to some embodiments. As shown in FIG. 1B, multi-lumen catheter 200 comprises an outer cross-sectional dimension D1, fluid lumen 120 comprises an inner cross-sectional dimension D2, and guide lumen 220 comprises an inner cross-sectional dimension D3, centered along a first axis 205. In some embodiments, Point A of the inner cross-sectional dimension D2 and Point B of the outer cross-sectional dimension D1 are separated by dimension D4. Likewise, in some embodiments, Point C of the inner cross-sectional dimension D3 and Point D of the outer cross-sectional dimension D1 are separated by dimension D5.

In some embodiments, a multi-lumen catheter comprises an outer cross-sectional dimension D1 of between 0.01 mm and 10 mm. In some embodiments, the outer cross-sectional dimension D1 is greater than or equal to 0.1 mm, greater than or equal to 0.5 mm, greater than or equal to 1.0 mm, greater than or equal to 1.5 mm, greater than or equal to 2 mm, greater than or equal to 2.5 mm, greater than or equal to 3 mm, greater than or equal to 3.5 mm, greater than or equal to 4 mm, greater than or equal to 4.5 mm, greater than or equal to 5 mm, greater than or equal to 5.5 mm, greater than or equal to 6 mm, greater than or equal to 6.5 mm, greater than or equal to 7 mm, greater than or equal to 7.5 mm, greater than or equal to 8 mm, greater than or equal to 8.5 mm, greater than or equal to 9 mm, greater than or equal to 9.5 mm, or greater than or equal to 10 mm. In some embodiments, the outer cross-sectional dimension D1 is less than or equal to 10 mm, less than or equal to 9.5 mm, less than or equal to 9 mm, less than or equal to 8.5 mm, less than or equal to 8 mm, less than or equal to 7.5 mm, less than or equal to 7 mm, less than or equal to 6.5 mm, less than or equal to 6 mm, less than or equal to 5.5 mm, less than or equal to 5 mm, less than or equal to 4.5 mm, less than or equal to 4 mm, less than or equal to 3.5 mm, less than or equal to 3 mm, less than or equal to 2.5 mm, less than or equal to 2.0 mm, less than or equal to 1.5 mm, less than or equal to 1 mm, less than or equal to 0.5 mm, or less than or equal to 0.1 mm. Combinations of the above cited ranges are also possible in some embodiments. For example, in some embodiments, the outer cross-sectional dimension D1 is greater than or equal to 0.1 mm, or less than or equal to 10 mm.

In some embodiments, a multi-lumen catheter comprises an outer cross-sectional dimension D1 of between 2 mm and 3 mm. In some embodiments, the outer cross-sectional dimension D1 is greater than or equal to 2.1 mm, greater than or equal to 2.2 mm, greater than or equal to 2.3 mm, greater than or equal to 2.4 mm, greater than or equal to 2.5 mm, greater than or equal to 2.6 mm, greater than or equal to 2.7 mm, greater than or equal to 2.8 mm, greater than or equal to 2.9 mm, or greater than or equal to 3.0 mm. In some embodiments, the outer cross-sectional dimension D1 is less than or equal to 3.0 mm, less than or equal to 2.9 mm, less than or equal to 2.8 mm, less than or equal to 2.7 mm, less than or equal to 2.6 mm, less than or equal to 2.5 mm, less than or equal to 2.4 mm, less than or equal to 2.3 mm, less than or equal to 2.2. mm, less than or equal to 2.1 mm, or less than or equal to 2.0 mm. Combinations of the above cited ranges are also possible in some embodiments. For example, in some embodiments, the outer cross-sectional dimension D1 is greater than or equal to 2 mm, or less than or equal to 3 mm. In some embodiments, the outer cross-sectional dimension D1 of the multi-lumen catheter is about 2.5 mm.

In some embodiments, a multi-lumen catheter comprises a fluid lumen comprising an inner cross-sectional dimension D2 of between 100 microns and 10 mm. In some embodiments, the inner cross-sectional dimension D2 is greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm. In some embodiments, the inner cross-sectional dimension D2 is less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns. Combinations of the above cited ranges are also possible in some embodiments. For example, in some embodiments, the inner cross-sectional dimension D2 is greater than or equal to 100 microns or less than or equal to 10 mm.

In some embodiments, a multi-lumen catheter comprises a fluid lumen comprising an inner cross-sectional dimension D2 of between 0.3 mm and 0.6 mm. In some embodiments, the inner cross-sectional dimension D2 is greater than or equal to 0.3 mm, greater than or equal to 0.4 mm, greater than or equal to 0.5 mm, or greater than or equal to 0.6 mm. In some embodiments, the inner cross-sectional dimension D2 is less than or equal to 0.6 mm, less than or equal to 0.5 mm, less than or equal to 0.4 mm, or less than or equal to 0.3 mm. Combinations of the above cited ranges are also possible in some embodiments. For example, in some embodiments, the inner cross-sectional dimension D2 is greater than or equal to 0.3 mm, or less than or equal to 0.6 mm. In some embodiments, the inner cross-sectional dimension D2 is about 0.5 mm.

In some embodiments, a multi-lumen catheter comprises a guide lumen comprising an inner cross-sectional dimension D3 of between 100 microns and 10 mm. In some embodiments, the inner cross-sectional dimension D3 is greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm. In some embodiments, the inner cross-sectional dimension D3 is less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns. Combinations of the above cited ranges are also possible in some embodiments. For example, in some embodiments, the inner cross-sectional dimension D3 is greater than or equal to 100 microns or less than or equal to 10 mm.

In some embodiments, a multi-lumen catheter comprises a guide lumen comprising an inner cross-sectional dimension D3 of between 1 mm and 2 mm. In some embodiments, the inner cross-sectional dimension D3 is greater than or equal to 1 mm, greater than or equal to 1.1 mm, greater than or equal to 1.2 mm, greater than or equal to 1.3 mm, greater than or equal to 1.4 mm, greater than or equal to 1.5 mm, greater than or equal to 1.6 mm, greater than or equal to 1.7 mm, greater than or equal to 1.8 mm, greater than or equal to 1.9 mm, or greater than or equal to 2.0 mm. In some embodiments, the inner cross-sectional dimension D3 is less than or equal to 2.0 mm, less than or equal to 1.9 mm, less than or equal to 1.8 mm, less than or equal to 1.7 mm, less than or equal to 1.6 mm, less than or equal to 1.5 mm, less than or equal to 1.4 mm, less than or equal to 1.3 mm, less than or equal to 1.2 mm, less than or equal to 1.1 mm, less than or equal to 1.0 mm. Combinations of the above cited ranges are also possible in some embodiments. For example, in some embodiments, the inner cross-sectional dimension D3 is greater than or equal to 1.0 mm, or less than or equal to 2.0 mm. In some embodiments, the inner cross-sectional dimension D3 is about 1.3 mm.

In some embodiments, Point A of the inner cross-sectional dimension D2 and Point B of the outer cross-sectional dimension D1 are separated by dimension D4. In some embodiment dimension D4 is between 100 microns and 10 mm. In some embodiments, dimension D4 is greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm. In some embodiments, dimension D4 is less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns. Combinations of the above cited ranges are also possible in some embodiments. For example, in some embodiments, dimension D4 is greater than or equal to 100 microns or less than or equal to 10 mm.

In some embodiments, Point A of the inner cross-sectional dimension D2 and Point B of the outer cross-sectional dimension D1 are separated by dimension D4. In some embodiment dimension D4 is between 0.10 mm and 0.4 mm. In some embodiments, dimension D4 is greater than or equal to 0.10 mm, greater than or equal to 0.15 mm, greater than or equal to 0.20 mm, greater than or equal to 0.25 mm, greater than or equal to 0.30 mm, greater than or equal to 0.35 mm, or greater than or equal to 0.40 mm. In some embodiments, dimension D4 is less than or equal to 0.40 mm, less than or equal to 0.35 mm, less than or equal to 0.30 mm, less than or equal to 0.25 mm, less than or equal to 0.20 mm, less than or equal to 0.15 mm, or less than or equal to 0.10 mm. Combinations of the above cited ranges are also possible in some embodiments. For example, in some embodiments, dimension D4 is greater than or equal to 0.10 mm, or less than or equal to 0.20 mm. In some embodiments, the dimension D4 is about 0.25 mm.

In some embodiments, Point C of the inner cross-sectional dimension D3 and Point D of the outer cross-sectional dimension D1 are separated by dimension D5. In some embodiment dimension D5 is between 100 micron and 10 mm. In some embodiments, dimension D5 is greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm. In some embodiments, dimension D5 is less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns. Combinations of the above cited ranges are also possible in some embodiments. For example, in some embodiments, dimension D5 is greater than or equal to 100 microns or less than or equal to 10 mm.

In some embodiments, Point C of the inner cross-sectional dimension D3 and Point D of the outer cross-sectional dimension D1 are separated by dimension D5. In some embodiment dimension D5 is between 0.10 mm and 0.4 mm. In some embodiments, dimension D5 is greater than or equal to 0.10 mm, greater than or equal to 0.15 mm, greater than or equal to 0.20 mm, greater than or equal to 0.25 mm, greater than or equal to 0.30 mm, greater than or equal to 0.35 mm, or greater than or equal to 0.40 mm. In some embodiments, dimension D5 is less than or equal to 0.40 mm, less than or equal to 0.35 mm, less than or equal to 0.30 mm, less than or equal to 0.25 mm, less than or equal to 0.20 mm, less than or equal to 0.15 mm, or less than or equal to 0.10 mm. Combinations of the above cited ranges are also possible in some embodiments. For example, in some embodiments, dimension D5 is greater than or equal to 0.10 mm, or less than or equal to 0.20 mm. In some embodiments, the dimension D5 is about 0.25 mm.

While FIG. 1B depicts the various dimensions (e.g., D1, D2, D3, D4, D5) as being in a relative position to one another, those of ordinary skill in the art would understand that the disclosure is not so limited and other arrangements and configurations are also possible. For example, while FIG. 1B depicts fluid lumen 210 and guide lumen 220 generally aligned along first axis 205, fluid lumen 210, guide lumen 220, or both, may be positioned such that their central point is not on the first axis 205. Those of ordinary skill in the art would understand, based upon the teachings of this specification, that fluid lumen 210 and guide lumen 220 may be positioned at any suitable location within catheter 200.

FIG. 1C shows a side view of exemplary multi-lumen catheter 300, according to some embodiments. As shown in FIG. 1C, the multi-lumen catheter has length of dimension D6. The multi-lumen catheter further comprises one or more graduations 310 (e.g., markings). Those of skill in the art will appreciate that such graduations may be useful, for example, for ensuring the catheter is placed at the appropriate depth. The distance between adjacent gradations may be any suitable distance known to those of skill in the art. In some embodiments, the distance between adjacent gradations is about 10 millimeters (mm). In some embodiments, the one or more graduations 310 is between 0 mm to 200 mm in 10 mm increments.

In some embodiments, the multi-lumen catheter has length of dimension D6 of between 50 mm and 300 mm. In some embodiments, the length of dimension D6 is greater than or equal to 50 mm, greater than or equal to 75 mm, greater than or equal to 100 mm, greater than or equal to about 125 mm, greater than or equal to 150 mm, greater than or equal to 175 mm, greater than or equal to 200 mm, greater than or equal to 225 mm, greater than or equal to 250 mm, greater than or equal to 275 mm, or greater than or equal to 300 mm. In some embodiments, the dimension D6 is less than or equal to 300 mm, less than or equal to 275 mm, less than or equal to 250 mm, less than or equal to 225 mm, less than or equal to 200 mm, less than or equal to 175, less than or equal to 150 mm, less than or equal to 125 mm, less than or equal to 100 mm, less than or equal to 75 mm, or less than or equal to 50 mm. Combinations of the above cited ranges are also possible in some embodiments. For example, in some embodiments, the length of dimension D6 is greater than or equal to 50 mm or less than or equal to 300 mm.

In some embodiments, the multi-lumen catheter has length of dimension D6 of between 150 mm and 250 mm. In some embodiments, the length of dimension D6 is greater than or equal to 150 mm, greater than or equal to 160 mm, greater than or equal to 170 mm, greater than or equal to about 180 mm, greater than or equal to 190 mm, greater than or equal to 200 mm, greater than or equal to 210 mm, greater than or equal to 220 mm, greater than or equal to 230 mm, greater than or equal to 240 mm, or greater than or equal to 250 mm. In some embodiments, the dimension D6 is less than or equal to 250 mm, less than or equal to 240 mm, less than or equal to 230 mm, less than or equal to 220 mm, less than or equal to 210 mm, less than or equal to 200, less than or equal to 190 mm, less than or equal to 180 mm, less than or equal to 170 mm, less than or equal to 160 mm, or less than or equal to 150 mm. Combinations of the above cited ranges are also possible in some embodiments. For example, in some embodiments, the length of dimension D6 is greater than or equal to 150 mm, or less than or equal to 250 mm. In some embodiments, the length of dimension D6 is about 200 mm.

In some embodiments, the catheters described herein may have a suitable aspect ratio. As would be understood by those of ordinary skill in the art, the aspect ratio generally increases as the device increases in length and decreases in width. Artisans will immediately appreciate that all ranges and values between the explicitly stated bounds are contemplated, with, e.g., any of the following being available as an upper or lower limit: 3:1, 4:1, 5:1, 6:1, 7:1, 8:1, 9:1, 10:1, 50:1, 100:1, 1000:1. A high aspect ratio is highly advantageous for certain devices, e.g., many types of catheters. In principle, a thin tube could be manufactured e.g., via continuous extruded without limitation as to length. Such devices include, e.g., tubes, rods, cylinders, and cross-sections with square, polygonal, or round profiles. One or more lumens may be provided in any of the same. The catheters may be made of a single material, essentially a single material, or with a plurality of materials including the various materials described below and/or may include one or more of a reinforcing material, a fiber, a wire, a braided material, braided wire, braided plastic fibers, or the like.

For example, the multi-lumen catheter may be comprised of any suitable material known to those of skill in the art, such as for example, silicone elastomer. In some embodiments, the multi-lumen catheter comprises a medical grade elastomer. Non-limiting embodiments include, for example, poly(1,1,2,2-tetraflurorethylene) (e.g., Teflon), ethylene propylene diene rubber (EPDM), perfluoroelastomers (e.g., Isolast® Plus J9515, J9516, J9538 FFKM), fluoroelastomers (e.g., FKM), and acrylonitrile-butadiene rubber (e.g., NBR and HNBR). In some embodiments, the plug comprises an adhesive silicone.

FIGS. 1D and 1E show a top-down view and side view of exemplary tube 400, for example, as shown in FIG. 1A (e.g., see tube 150 in FIG. 1A). In some embodiments, the tube has a length of dimension D9, an outer cross-sectional dimension D7, and an inner cross-sectional dimension D8. In some embodiments, the length of dimension D9 is between 5 and 30 mm. In some embodiments, the tube length of dimension D9 is greater than or equal to 5 mm, is greater than or equal to 6 mm, is greater than or equal to 7 mm, is greater than or equal to 8 mm, is greater than or equal to 9 mm, is greater than or equal to 10 mm, greater than or equal to 12 mm, greater than or equal to 15 mm, greater than or equal to 17 mm, greater than or equal to 20 mm, greater than or equal to 22 mm, greater than or equal 25 mm, greater than or equal to 27 mm, or greater than or equal to 30 mm. In some embodiments, the tube length of dimension D9 is less than or equal to 30 mm, less than or equal to 27 mm, less than or equal to 25 mm, less than or equal to 22 mm, less than or equal to 20 mm, less than or equal to 17 mm, less than or equal to 15 mm, less than or equal to 12 mm, less than or equal to 10 mm, less than or equal to 9 mm, less than or equal to 8 mm, less than or equal to 7 mm, less than or equal to 6 mm, or less than or equal to 5 mm. Combinations of the above cited ranges are also possible in some embodiments. For example, in some embodiments, the tube length of dimension D9 is greater than or equal to 5 mm, or less than or equal to 30 mm. In some embodiments, the tube length of dimension D9 is about 20 mm.

In some embodiments, a tube has an outer cross-sectional dimension D7 of between 10 microns mm and 1000 microns. In some embodiments, the tube has an outer cross-sectional diameter D7 of greater than or equal to 10 microns, greater than or equal to 25 microns, greater than or equal to 50 microns, greater than or equal to 75 microns, greater than or equal to 100 microns, greater than or equal to 125 microns, greater than or equal to 150 microns, greater than or equal to 175 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1000 microns. In some embodiments, dimension D7 is less than or equal to 1000 microns, is less than or equal to 900 microns, is less than or equal to 800 microns, is less than or equal to 700 microns, is less than or equal to 600 microns, is less than or equal to 500 microns, is less than or equal to 400 microns, is less than or equal to 300 microns, is less than or equal to 200 microns, is less than or equal to 175 microns, is less than or equal to 150 microns, is less than or equal to 125 microns, is less than or equal to 100 microns, is less than or equal to 75 microns, is less than or equal to 50 microns, is less than or equal to 25 microns, or is less than or equal to 10 microns. Combinations of the above cited ranges are also possible in some embodiments. For example, in some embodiments, dimension D7 is greater than or equal to 10 microns or less than or equal to 1000 microns.

In some embodiments, a tube has an outer cross-sectional dimension D7 of between 0.1 mm and 1.0 mm. In some embodiments, the outer cross-sectional dimension D7 of the tube is greater than or equal to 0.1 mm, greater than or equal to 0.2 mm, greater than or equal to 0.3 mm, greater than or equal to 0.4 mm, greater than or equal to 0.5 mm, greater than or equal to 0.6 mm, greater than or equal to 0.7 mm, greater than or equal to 0.8 mm, greater than or equal to 0.9 mm, or greater than or equal to 1.0 mm. In some embodiments, the outer cross-sectional dimension D7 of the tube is less than or equal to 1.0 mm, less than or equal to 0.9 mm, less than or equal to 0.8 mm, less than or equal to 0.7 mm, less than or equal to 0.6 mm, less than or equal to 0.5 mm, less than or equal to 0.4 mm, less than or equal to 0.3 mm, less than or equal to 0.2 mm, or less than or equal to 0.1 mm. Combinations of the above cited ranges are also possible in some embodiments. For example, in some embodiments, the outer cross-sectional dimension D7 of the tube is greater than or equal to 0.1 mm, or less than or equal to 1.0 mm.

In some embodiments, a tube has an inner cross-sectional dimension D8 of between 10 microns mm and 200 microns. In some embodiments, the tube has an inner cross-sectional diameter D8 of is greater than or equal to 10 microns, greater than or equal to 25 microns, greater than or equal to 50 microns, greater than or equal to 75 microns, greater than or equal to 100 microns, greater than or equal to 125 microns, greater than or equal to 150 microns, greater than or equal to 175 microns, or greater than or equal to 200 microns. In some embodiments, dimension D8 is less than or equal to 200 microns, is less than or equal to 175 microns, is less than or equal to 150 microns, is less than or equal to 125 microns, is less than or equal to 100 microns, is less than or equal to 75 microns, is less than or equal to 50 microns, is less than or equal to 25 microns, or is less than or equal to 10 microns. Combinations of the above cited ranges are also possible in some embodiments. For example, in some embodiments, dimension D8 is greater than or equal to 50 microns or less than or equal to 200 microns.

In some embodiments, a tube has an inner cross-sectional dimension D8 of between 0.1 mm and 0.2 mm. In some embodiments, the inner cross-sectional dimension D8 of the tube is greater than or equal to 0.11 mm, greater than or equal to 0.12 mm, greater than or equal to 0.13 mm, greater than or equal to 0.14 mm, greater than or equal to 0.15 mm, greater than or equal to 0.16 mm, greater than or equal to 0.17 mm, greater than or equal to 0.18 mm, greater than or equal to 0.19 mm, or greater than or equal to 0.20 mm. In some embodiments, the inner cross-sectional dimension D8 of the tube is less than or equal to 0.2 mm, less than or equal to 0.19 mm, less than or equal to 0.18 mm, less than or equal to 0.17 mm, less than or equal to 0.16 mm, less than or equal to 0.15 mm, less than or equal to 0.14 mm, less than or equal to 0.13 mm, less than or equal to 0.12. mm, less than or equal to 0.11 mm, or less than or equal to 0.10 mm. Combinations of the above cited ranges are also possible in some embodiments. For example, in some embodiments, the inner cross-sectional dimension D8 of the tube is greater than or equal to 0.1 mm or less than or equal to 0.2 mm. In some embodiments, the tube has an inner cross-sectional dimension D8 of about 0.15 mm.

In some embodiments, a tube is sized and configured to be positioned within at least a portion of a fluidic lumen of a multi-lumen catheter. In some embodiments, the tube is configured to be reversibly placed within at least a portion of the fluidic lumen. In other embodiments, however, the tube is configured to be permanently positioned within at least a portion of the fluidic lumen. In some embodiments, the tube extends beyond a distal end of the multi-lumen catheter by between 1 mm and 25 mm. In some embodiments, the tube extends beyond the distal end of the multi-lumen catheter by a distance greater than or equal to 1 mm, greater than or equal to 2 mm, greater than or equal to 3 mm, greater than or equal to 4 mm, greater than or equal to 5 mm, greater than or equal to 7 mm, greater than or equal to 10 mm, greater than or equal to 12 mm, greater than or equal to 15 mm, greater than or equal to 17 mm, greater than or equal to 20 mm, greater than or equal to 21 mm, greater than or equal to 22 mm, greater than or equal to 23 mm, greater than or equal to 24 mm, or greater than or equal to 25 mm,. In some embodiments, the tube extends beyond the distal end of the multi-lumen catheter by a distance less than or equal to 25 mm, less than or equal to 24 mm, less than or equal to 23 mm, less than or equal to 22 mm, less than or equal to 21 mm, less than or equal to 20 mm, less than or equal to 17 mm, less than or equal to 15 mm, less than or equal to 12 mm, less than or equal to 10 mm, less than or equal to 7 mm, less than or equal to 5 mm, less than or equal to 4 mm, less than or equal to 3 mm, less than or equal to 2 mm, or less than or equal to 1 mm. Combinations of the above cited ranges are also possible in some embodiments. For example, in some embodiments, the tube extends beyond the distal end of the multi-lumen catheter by a distance of greater than or equal to 1 mm or less than or equal to 25 mm. In some embodiments, the tube extends beyond the distal end of the multi-lumen catheter by a distance of about 10 mm.

The tube may be comprised of any suitable material known to the skilled artisan. Exemplary embodiments include, but are not limited to, polymers, glass, ceramics, metals, or the like. In some embodiments, the tube comprises fused silica (e.g., quartz glass or fused quartz). In other embodiments the tube comprises polyether ether ketone (PEEK).

As shown in FIG. 1A, in some embodiments, plug 140 is sized and configured to be positioned within distal portion 160 of guide lumen 120 of exemplary multi-lumen catheter 100. Without wishing to be bound by any particular theory, it is generally believed that positioning plug 140 within distal portion 160 of the guide lumen 120 prevents body fluid (e.g., spinal fluid, interstitial fluid) from infiltrating the guide lumen, for example, during placement of the device within a tissue or cavity of a subject (e.g., tumor tissue, spinal cavity, or brain ventricle or other cavity). Plug 140 also prevents backflow into the guide lumen 120 following delivery of a fluid through the fluid lumen 110 of the multi-lumen catheter 100 (e.g., via convention enhanced delivery or other bulk flow delivery method). Such configurations are useful, for example, for medical applications where a target fluid (e.g., a chemotherapeutic agent) needs to be delivered to a target tissue (e.g., brain tissue) using bulk flow (e.g., convection enhanced delivery (CED)). As discussed elsewhere herein, bulk flow refers to the continuous infusion of a target fluid (e.g., a chemotherapeutic agent) into a target tissue (e.g., brain tumor tissue) using, for example, a fusion pump. This creates pressure gradients that push the target fluid into the tissue interstitial space, which in turn, displaces the extracellular fluid. Plug 140 in guide lumen 120 prevents back flow of the displaced extracellular fluid into the guide lumen 120, and thus, serves to maximize drug delivery at the target site and may further improve diffusion of the drug within the target tissue. In some embodiments, plug 140 has a length of between 2 mm and 4 mm. In some embodiments, plug 140 has a length of about 3 mm.

The plug may be comprised of any suitable material known to those of skill in the art, such as for example, silicone. In some embodiments, the plug comprises a medical grade elastomer. Non-limiting embodiments include, for example, ethylene propylene diene rubber (EPDM), perfluoroelastomers (e.g., Isolast® Plus J9515, J9516, J9538 FFKM), fluoroelastomers (e.g., FKM), and acrylonitrile-butadiene rubber (e.g., NBR and HNBR). In some embodiments, the plug comprises an adhesive silicone. In some embodiments, the plug comprises polytetrafluoroethylene (PFTE).

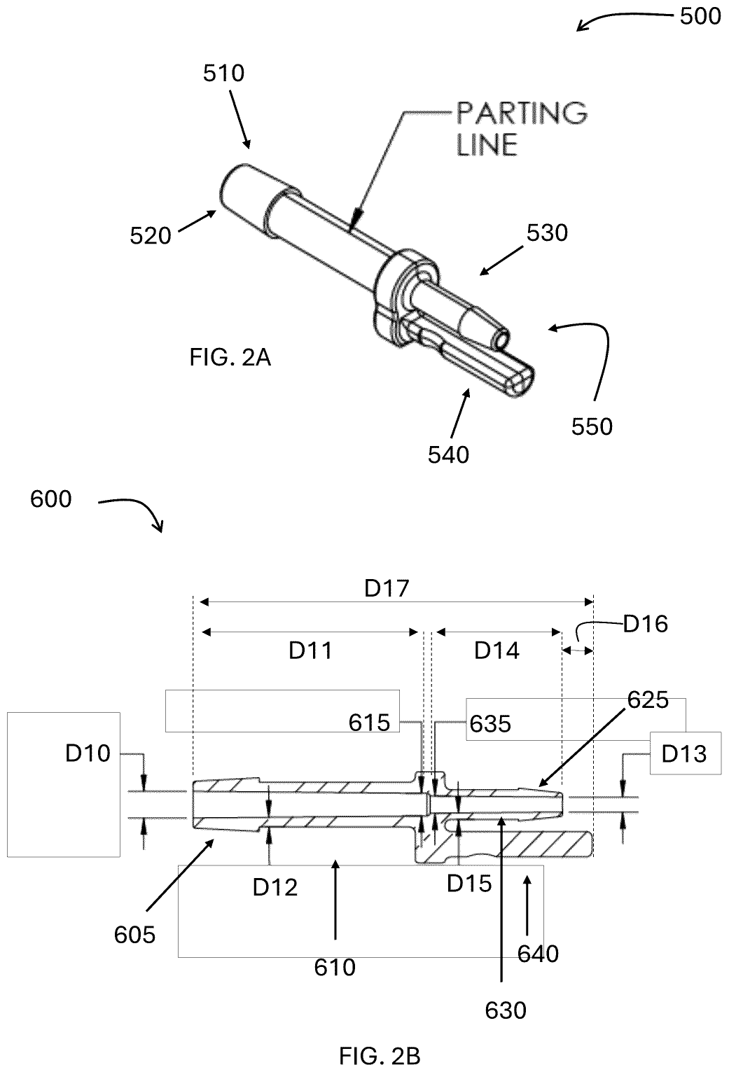

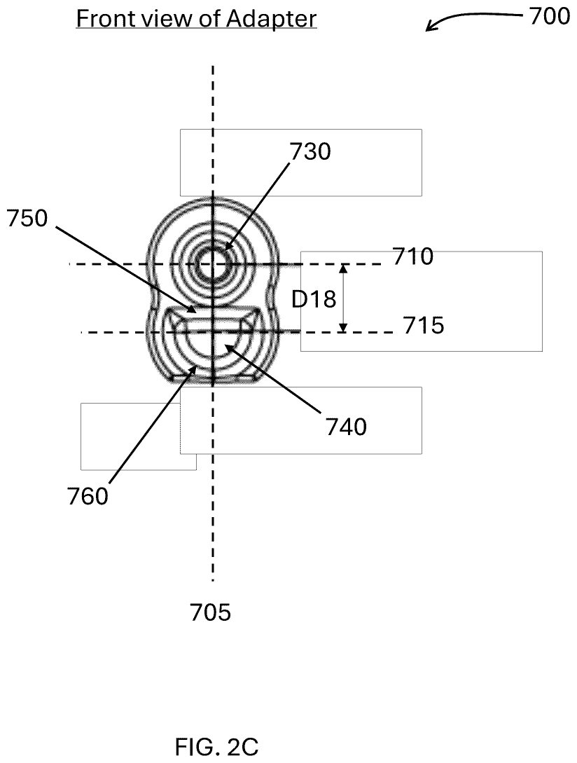

FIG. 2A shows an off-set view of an exemplary adapter 500 as contemplated herein. In some embodiments, the adapter comprises connector port 510 positioned at a first end 520 of exemplary adapter 500. In some embodiments, exemplary adapter 500 comprises fluid port 530 and stylet port 540 positioned at a second end 550 of the adapter. As used herein, the term “adapter” is synonymous with the terms “fitted adapter” and “fluid adapter.” As used herein, the term “connector port” is synonymous with the term “first plug.” In some embodiments, the term “fluid port” is synonymous with the term “second plug” and the term “stylet port” is synonymous with the term “third plug.”

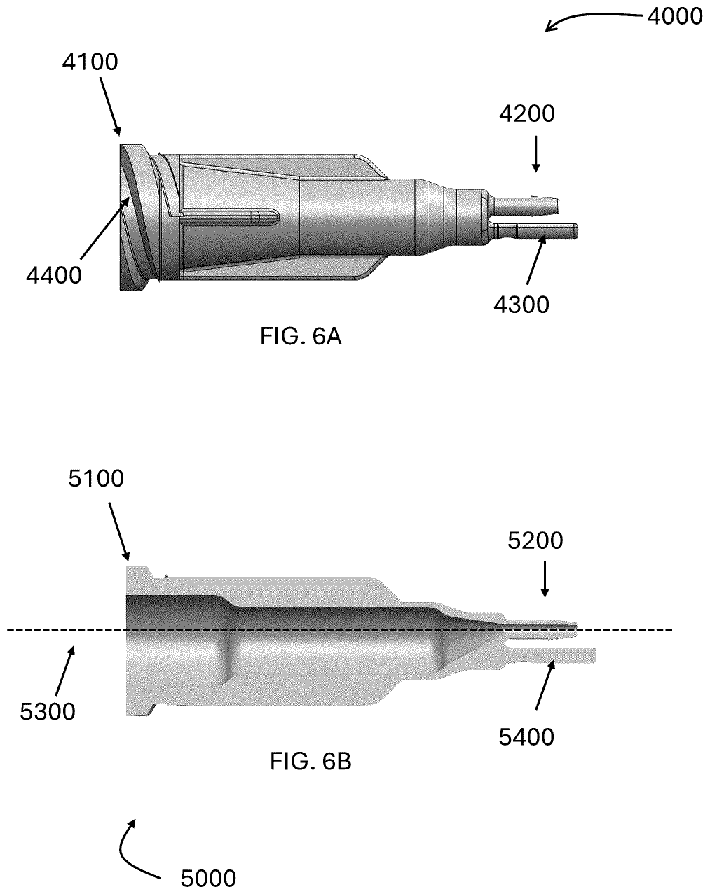

In some embodiments, fluid port 530 of exemplary adapter 500 is in fluidic communication with connector port 510 of the adapter (e.g., via a lumen, described in FIG. 2B and FIG. 6B below). In some embodiments, stylet port 540 of exemplary adapter 500 is in fluidic communication with connector port 510. In some embodiments, connector port 510, fluid port 530, and stylet port 540 are in all in fluidic communication. In some embodiments, the connector of the adapter has an inner diameter, D10, that is larger than inner diameter D11 of the fluid port of the adapter.

In some embodiments, stylet port 540 is proximate fluid port 530 (e.g., is disposed directly beneath the fluid port). In some embodiments, style port 540 comprises a first surface and a second surface. In some instances, the first surface and second surface have different curvatures (e.g., a first surface is flat and a second surface is curved). In some embodiments, at least a portion of the first surface extends beyond a distal portion of the fluid port (e.g., the stylet port is longer than then fluid port).

FIG. 2B shows a cross-sectional view of an exemplary adapter 600 as contemplated herein. In some embodiments, connector port 610 of adapter 600 comprises a connector port lumen 615 having diameter D10 and length D11. In some embodiments, connector port 610 has a wall thickness of D12 (e.g., the distance between the inner wall of the connector port lumen and the outer wall of the connector port). In some embodiments, one or more sides 620 of connector port 605 are tapered.

In some embodiments, fluid port 630 of exemplary adapter 600 comprises a fluid port lumen 635 having diameter D13 and length D14. In some embodiments, the fluid port 630 has a wall thickness of D15 (e.g., the distance between the inner wall of the fluid port lumen and the outer wall of the fluid port). In some embodiments, one or more sides 625 of fluid port 630 are tapered.

In some embodiments, an inner diameter of a connector port lumen of an adapter is larger than the inner diameter of the fluid port lumen of the adapter. In some embodiments, the fluid port lumen is in fluidic communication with the connector port lumen (e.g., the connector port is in fluidic communication with the fluid port via the lumens). In some embodiments, stylet port 640 of exemplary adapter 600 comprises a stylet port lumen (not shown in FIG. 2B). In some embodiments, connector port lumen 615 is in fluidic communication with fluid port lumen 635 and/or the stylet port lumen.

In some embodiments, an adapter comprises a connector port lumen having length D11 of between 5 mm and 20 mm in length. In some embodiments, the connector port lumen has a length of greater than or equal to 5 mm, greater than or equal to 5.5 mm, greater than or equal to 6 mm, greater than or equal to 6.5 mm, greater than or equal to 7.0 mm, greater than or equal to 7.5 mm, greater than or equal to 8.0 mm, greater than or equal to 8.5 mm, greater than or equal to 9 mm, greater than or equal to 9.5 mm, greater than or equal to 10 mm greater than or equal to 12 mm, greater than or equal to 14 mm, greater than or equal to 16 mm, greater than or equal to 18 mm, or greater than or equal to 20 mm. In some embodiments, the connector port lumen has a length of less than or equal to 20 mm, less than or equal to 18 mm, less than or equal to 16 mm, less than or equal to 14 mm, less than or equal to 12 mm, less than or equal to 10 mm, less than or equal to 9.5 mm, less than or equal to 9 mm, less than or equal to 8.5 mm, less than or equal to 8 mm, less than or equal to 7.5 mm, less than or equal to 7 mm, less than or equal to 6.5 mm, less than or equal to 6 mm, less than or equal to 5.5 mm, or less than or equal to 5 mm. Combinations of the above recited ranges are also possible, in some embodiments. For example in some embodiments, the connector port lumen has length D11 of greater than or equal to 5 mm and less than or equal to 20 mm.

In some embodiments, an adapter comprises a connector port lumen having length D11 of between 5 mm and 15 mm. In some embodiments, the connector port has a length of greater than or equal to 5 mm, greater than or equal to 6 mm, greater than or equal to greater than 7 mm, greater than or equal to 8 mm, greater than or equal to 9 mm, greater than or equal to 10 mm, greater than or equal to 11 mm, greater than or equal to 12 mm, greater than or equal to 13 mm, greater than or equal to 14 mm, greater than or equal to 15 mm. In some embodiments, the connector port has a length of less than or equal to 15 mm, less than or equal to 14 mm, less than or equal to 13 mm, less than or equal to 12 mm, less than or equal to 11 mm, less than or equal to 10 mm, less than or equal to 9 mm, less than or equal to 8 mm, less than or equal to 7 mm, less than or equal to 6 mm, or less than or equal to 5 mm. Combinations of the above recited ranges are also possible, in some embodiments. For example in some embodiments, the connector port lumen has length D11 of greater than or equal to 5 mm and less than or equal to 15 mm.

In some embodiments, an adapter comprises a connector port lumen having length D11 of between 7.00 mm and 8.00 mm. In some embodiments, the connector port has a length of greater than or equal to 7.00 mm, greater than or equal to 7.25 mm, greater than or equal to greater than 7.55 mm, greater than or equal to 7.75 mm, or greater than or equal to 8.00 mm. In some embodiments, the connector port has a length of less than or equal to 8.00 mm, length than or equal to 7.75 mm, length than or equal to greater than 7.55 mm, length than or equal to 7.25 mm, or length than or equal to 8.00 mm. Combinations of the above recited ranges are also possible, in some embodiments. For example in some embodiments, the connector port lumen has length D11 of greater than or equal to 7 mm and less than or equal to 10 mm. Other combinations are also possible. For example, in some embodiments, the connector port lumen has length D11 of greater than or equal to 7 mm and less than or equal to 8 mm. In some embodiments, the connector port lumen has length D11 that is about 7.55 mm.

In some embodiments, an adapter comprises a connector port lumen having inner diameter D10 of between 10 microns mm and 2000 microns. In some embodiments, the connector port lumen has inner diameter D10 of greater than or equal to 10 microns, greater than or equal to 25 microns, greater than or equal to 50 microns, greater than or equal to 75 microns, greater than or equal to 100 microns, greater than or equal to 250 microns, greater than or equal to 500 microns, greater than or equal to 750 microns, greater than or equal to 1000 microns, greater than or equal to 1250 microns, greater than or equal to 1500 microns, greater than or equal to 1700 microns, greater than or equal to 2000 microns. In some embodiments, dimension D10 is less than or equal to 2000 microns, is less than or equal to 1700 microns, is less than or equal to 1500 microns, is less than or equal to 1250 microns, is less than or equal to 1000 microns, is less than or equal to 750 microns, is less than or equal to 500 microns, is less than or equal to 250 microns, is less than or equal to 100 microns, is less than or equal to 75 microns, is less than or equal to 50 microns, is less than or equal to 25 microns, is less than or equal to 10 microns. Combinations of the above cited ranges are also possible in some embodiments. For example, in some embodiments, dimension D10 is greater than or equal to 10 microns or less than or equal to 2000 microns.

In some embodiments, an adapter comprises a connector port lumen having inner diameter D10 of between 0.5 mm and 1.0 mm. In some embodiments, the connector port lumen has inner diameter D10 of greater than or equal to 0.5 mm, greater than or equal to 0.6 mm, greater than or equal to 0.7 mm, greater than or equal to 0.8 mm, greater than or equal to 0.9 mm, or greater than or equal to 1.0 mm. In some embodiments, the connector port lumen has inner diameter D10 of less than or equal to 1.0 mm, less than or equal to 0.9 mm, less than or equal to 0.8 mm, less than or equal to 0.7 mm, less than or equal to 0.6 mm, or less than or equal to 0.5 mm. Combinations of the above recited ranges are also possible in some embodiments. For example, in some embodiments, the connector port lumen has inner diameter D10 of greater than or equal to 0.5 mm and less than or equal to 1.0 mm. In some embodiments, the connector port lumen has inner diameter D10 of about 0.891 mm.

In some embodiments, an adapter comprises a connector port lumen having wall thickness D12 of between 10 microns mm and 2000 microns. In some embodiments, the adapter comprises a connector port lumen having wall thickness D12 of greater than or equal to 10 microns, greater than or equal to 25 microns, greater than or equal to 50 microns, greater than or equal to 75 microns, greater than or equal to 100 microns, greater than or equal to 250 microns, greater than or equal to 500 microns, greater than or equal to 750 microns, greater than or equal to 1000 microns, greater than or equal to 1250 microns, greater than or equal to 1500 microns, greater than or equal to 1700 microns, greater than or equal to 2000 microns. In some embodiments, dimension D12 is less than or equal to 2000 microns, is less than or equal to 1700 microns, is less than or equal to 1500 microns, is less than or equal to 1250 microns, is less than or equal to 1000 microns, is less than or equal to 750 microns, is less than or equal to 500 microns, is less than or equal to 250 microns, is less than or equal to 100 microns, is less than or equal to 75 microns, is less than or equal to 50 microns, is less than or equal to 25 microns, is less than or equal to 10 microns. Combinations of the above cited ranges are also possible in some embodiments. For example, in some embodiments, dimension D12 is greater than or equal to 10 microns or less than or equal to 2000 microns.

In some embodiments, an adapter comprises a connector port lumen having wall thickness D12 of between 0.2 mm and 0.4 mm. In some embodiments, the connector port lumen has wall thickness D12 of greater than or equal to 0.200 mm, greater than or equal to 0.225 mm, greater than or equal to 0.25 mm, greater than or equal to 0.275 mm, greater than or equal to 0.300 mm, greater than or equal to 0.325 mm, greater than or equal to 0.350 mm, greater than or equal to 0.375 mm, or greater than or equal to 0.400 mm. In some embodiments, the connector port lumen has wall thickness D12 of less than or equal to 0.400 mm, less than or equal to 0.375 mm, less than or equal to 0.350 mm, less than or equal to 0.350 mm, less than or equal to 0.325 mm, less than or equal to 0.300 mm, less than or equal to 0.275 mm, less than or equal to 0.250 mm, less than or equal to 0.225 mm, or less than or equal to 0.200 mm. Combinations of the above recited ranges are also possible in some embodiments. For example, in some embodiments, the connector port lumen has wall thickness D12 of greater than or equal to 0.2 mm and less than or equal to 0.4 mm. In some embodiments, the connector port lumen has wall thickness D12 of about 0.324 mm.

In some embodiments, an adapter comprises a fluid port lumen having length D14 of between 3 mm and 15 mm. In some embodiments, the fluid port lumen has length D14 of greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm, greater than or equal to 11 mm, greater than or equal to 12 mm, greater than or equal to 13 mm, greater than or equal to 14 mm, or greater than or equal to 15 mm. In some embodiments, the fluid port lumen has length D14 of less than or equal to 15 mm, less than or equal to 14 mm, less than or equal to 13 mm, less than or equal to 12 mm, less than or equal to 11 mm, less than or equal to 10 mm, less than or equal to 9 mm, less than or equal to 8 mm, less than or equal to 7 mm, less than or equal to 6 mm, less than or equal to 5 mm, less than or equal to 4 mm, or less than or equal to 3 mm. Combinations of the above recited ranges are also possible, in some embodiments. For example, in some embodiments, the fluid port lumen has length D14 of greater than or equal to 3 mm and less than or equal to 15 mm.

In some embodiments, an adapter comprises a fluid port lumen having length D14 of between 3 mm and 5 mm. In some embodiments, the fluid port lumen has length D14 of greater than or equal to 3 mm, greater than or equal to 3.25 mm, greater than or equal to 3.5 mm, greater than or equal to 3.75 mm, greater than or equal to 4.0 mm, greater than or equal to 4.25 mm, greater than or equal to 4.5 mm, greater than or equal to 4.75 mm, or greater than or equal to 5 mm. In some embodiments, the fluid port lumen has length D14 of less than or equal to 5 mm, less than or equal to 4.75 mm, less than or equal to 4.5 mm, less than or equal to 4.25 mm, less than or equal to 3.75 mm, less than or equal to 3.5 mm, less than or equal to 3.25 mm, or less than or equal to 3.0 mm. Combinations of the above recited ranges are also possible, in some embodiments. For example, in some embodiments, the fluid port lumen has length D14 of greater than or equal to 3 mm and less than or equal to 5 mm. In some embodiments, the fluid port lumen has length D14 of about 4 mm.

In some embodiments, an adapter comprises a fluid port lumen having inner diameter D13 of between 0.25 mm and 1 mm. In some embodiments, the fluid port lumen has inner diameter D13 of greater than or equal to 0.25 mm, greater than or equal to 0.30 mm, greater than or equal to 0.35 mm, greater than or equal to 0.4 mm, greater than or equal to 0.45 mm, or greater than or equal to 0.5 mm, greater than or equal to 0.55 mm, greater than or equal to 0.6 mm, greater than or equal to 0.65 mm, greater than or equal to 0.7 mm, greater than or equal to 0.75 mm or greater than or equal to 1.0 mm. In some embodiments, the fluid port lumen has inner diameter D13 of less than or equal to 1.0 mm, less than or equal to 0.75 mm, less than or equal to 0.70 mm, less than or equal to 0.65 mm, less than or equal to 0.60 mm, less than or equal to 0.55 mm, less than or equal to 0.5 mm, less than or equal to 0.45 mm, less than or equal to 0.40 mm, less than or equal to 0.35 mm, less than or equal to 0.3 mm, or less than or equal to 0.25 mm. Combinations of the above recited ranges are also possible in some embodiments. For example, in some embodiments, the fluid port lumen has inner diameter D13 of greater than or equal to 0.25 mm and less than or equal to 1.0 mm. In some embodiments, the fluid port lumen has inner diameter D13 of about 0.5 mm.

In some embodiments, an adapter comprises a fluid port lumen having wall thickness D15 of between 0.100 mm and 0.300 mm. In some embodiments, the fluid port lumen has wall thickness D15 of greater than or equal to 0.100 mm, greater than or equal to 0.125 mm, greater than or equal to 0.155 mm, greater than or equal to 0.175 mm, greater than or equal to 0.200 mm, greater than or equal to 0.225 mm, greater than or equal to 0.250 mm, greater than or equal to 0.2375 mm, or greater than or equal to 0.300 mm. In some embodiments, the fluid port lumen has wall thickness D15 of less than or equal to 0.300 mm, less than or equal to 0.275 mm, less than or equal to 0.250 mm, less than or equal to 0.250 mm, less than or equal to 0.225 mm, less than or equal to 0.200 mm, less than or equal to 0.175 mm, less than or equal to 0.150 mm, less than or equal to 0.125 mm, or less than or equal to 0.100 mm. Combinations of the above recited ranges are also possible in some embodiments. For example, in some embodiments, the fluid port lumen has wall thickness D15 of greater than or equal to 0.1 mm and less than or equal to 0.3 mm. In some embodiments, the fluid port lumen has wall thickness D15 of about 0.217 mm.

In some embodiments, one or more sides of the connector port and/or fluid port of the fluid adapter are tapered between 1° and 10° (degrees), relative to a first axis. In some embodiments, the one or more sides of the connector port and/or fluid port of the fluid adapter are tapered by an amount greater than or equal to 1°, greater than or equal to 2°, greater than or equal to 3°, greater than or equal to 4°, greater than or equal to 5°, greater than or equal to 6°, greater than or equal to 7°, greater than or equal to 8°, greater than or equal to 9°, greater than or equal to 10°, relative to a first axis. In some embodiments, the one or more sides of the connector port and/or fluid port of the fluid adapter are tapered by an amount less than or equal to 10°, less than or equal to 9°, less than or equal to 8°, less than or equal to 7°, less than or equal to 6°, less than or equal to 5°, less than or equal to 4°, less than or equal to 3°, less than or equal to 2°, less than or equal to 1°, relative to a first axis. Combinations of the above recited ranges are also possible in some embodiments. For example, in some embodiments, the one or more sides of the connector port and/or fluid port of the fluid adapter are tapered by greater than or equal to 1° and less than or equal to 10°. In some embodiments, the one or more sides of the connector port and/or fluid port of the fluid adapter are tapered by about 6°.

In some embodiments, stylet port 640 of exemplary adapter 600 extends past the end of fluid port 630 by a distance D16. In some embodiments, D16 is between 0.5 mm and 2 mm. In some embodiments, stylet port 640 of exemplary adapter 600 extends past the end of the fluid port 630 by a distance D16 of greater than or equal to 0.5 mm, greater than or equal to 0.75 mm, greater than or equal to 1.0 mm, greater than or equal to 1.25 mm, greater than or equal to 1.50 mm, greater than or equal to 1.75 mm, or greater than or equal to 2.0 mm. In some embodiments, stylet port 640 of exemplary adapter 600 extends past the end of fluid port 630 by a distance D16 of less than or equal to 2.0 mm, less than or equal to 1.75 mm, less than or equal to 1.5 mm, less than or equal to 1.25 mm, less than or equal to 1.0 mm, less than or equal to 0.75 mm, or less than or equal to 0.5 mm. Combinations of the above recited ranges are also possible in some embodiments. For example, in some embodiments, the stylet port of the adapter extends past the end of the fluid port of the adapter by a distance D16 of greater than or equal to 0.5 mm and less than or equal to 2 mm. In some embodiments, the stylet port of the adapter extends past the end of the fluid port of the adapter by a distance D16 of 1.0 mm.

The stylet port may have any suitable cross-sectional shape. In some embodiments, the shape may be any suitable cross-sectional shape including circular, oval, triangular, irregular, trapezoidal, square or rectangular, or the like. In certain embodiments, the stylet port may have a non-circular cross-sectional shape. In some embodiments, the stylet port may have a hemicircular cross-sectional shape. Other shapes are also possible.

In some embodiments, the cross-sectional shape and/or the cross-sectional dimension of the stylet port corresponds substantially to the cross-sectional shape and/or the cross-sectional dimension of the guide lumen. In some embodiments, the cross-sectional shape and/or the cross-sectional dimension of the stylet port does not correspond to the cross-sectional shape and/or the cross-sectional dimension of the guide lumen. For example, in some embodiments, the stylet port has a cross-sectional dimension substantially similar to D3 but a cross-sectional shape (e.g., hemicircular) different from a cross-sectional shape of the guide lumen (e.g., circular).

For example, in some embodiments, the stylet port comprises a first surface and a second surface, the first surface and second surface having different curvatures, wherein at least a portion of the first surface extends beyond a distal portion of the fluid port, as described in more detail herein.

In some embodiments, an adapter has overall length D17 of between 5 mm and 50 mm. In some embodiments, the adapter has overall length D17 of greater than or equal to 5 mm, greater than or equal to 10 mm, greater than or equal to 15 mm, greater than or equal to 20 mm, greater than or equal to 25 mm, or greater than or equal to 30 mm, greater than or equal to 35 mm, greater than or equal to 40 mm, greater than or equal to 45 mm, or greater than or equal to 50 mm. In some embodiments, the adapter has overall length D17 of less than or equal to 50 mm, less than or equal to 45 mm, less than or equal to 40 mm, less than or equal to 35 mm, less than or equal to 30 mm, less than or equal to 25 mm, less than or equal to 20 mm, less than or equal to 15 mm, less than or equal to 10 mm, or less than or equal to 5 mm. Combinations of the above recited ranges are also possible in some embodiments. For example, in some embodiments, the adapter has overall length D17 of greater than or equal to 5 mm and less than or equal to 50 mm.

In some embodiments, an adapter has overall length D17 of between 12 mm and 14 mm. In some embodiments, the adapter has overall length D17 of greater than or equal to 12.00 mm, greater than or equal to 12.25 mm, greater than or equal to 12.50 mm, greater than or equal to 12.75 mm, greater than or equal to 13.00 mm, or greater than or equal to 13.25 mm, greater than or equal to 13.50 mm, greater than or equal to 13.75 mm, or greater than or equal to 14.00 mm. In some embodiments, the adapter has overall length D17 of less than or equal to 14.00 mm, less than or equal to 13.75 mm, less than or equal to 13.50 mm, less than or equal to 13.25 mm, less than or equal to 13.00 mm, less than or equal to 12.75 mm, less than or equal to 12.50 mm, less than or equal to 12.25 mm, or less than or equal to 12.00 mm. Combinations of the above recited ranges are also possible in some embodiments. For example, in some embodiments, the adapter has overall length D17 of greater than or equal to 12 mm and less than or equal to 14.4 mm. In some embodiments, the adapter has overall length D17 of about 13.55 mm.

FIG. 2C shows a front view of an exemplary adapter 700, according to some embodiments. As illustrated in FIG. 2C, in some embodiments, a fluid port 730 is centered at the intersection of a first axis 705 and a second axis 710; and a stylet port 740 is centered at the intersection of the first axis 705 and a third axis 715, wherein the second axis 710 and third axis 715 are perpendicular (e.g., 90 degrees) to the first axis 705. In some embodiments, the center point of the fluid port 730 is separated by a distance D18 from the center point of the style port 740. In some embodiments, distance D18 is between 0.5 mm and 2 mm. For example, in some embodiments, the distance D18 is greater than or equal to 0.5 mm, greater than or equal to 0.75 mm, greater than or equal to 1.0 mm, greater than or equal to 1.25 mm, greater than or equal to 1.5 mm, greater than or equal to 1.75 mm, or greater than or equal to 2.0 mm. In some embodiments, the distance D18 is less than or equal to 2.0 mm, less than or equal to 1.75 mm, less than or equal to 1.50 mm, less than or equal to 1.25 mm, less than or equal to 1.00 mm, less than or equal to 0.75 mm, or less than or equal to 0.5 mm. Combinations of the above recited ranges are also possible in some embodiments. For example, in some embodiments, the distance D18 is greater than or equal to 0.5 mm and less than or equal to 2 mm. In some embodiments, the distance D18 is about 1.10 mm.

FIG. 2C also illustrates stylet port 740 comprising a first surface 750 and a second surface 760 opposite the first surface 750. In some embodiments, the first surface 750 and second surface 760 have different curvatures, relative to a first axis 705 and third axis 715.

In some embodiments, the first surface has zero curvature (e.g., it is parallel to the third axis 715). In some embodiments, the second surface has a radius of curvature of between 100 microns and 10 mm. In some embodiments, the second surface has a radius of curvature of between 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm. In some embodiments, the second surface has a radius of curvature of less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns. Combinations of the above cited ranges are also possible in some embodiments. For example, in some embodiments, the second surface has a radius of curvature of greater than or equal to 100 microns or less than or equal to 10 mm.

In some embodiments, a connector port of an adapter is configured to connect to a fluid adapter port of a reservoir. In some embodiments, a fluid port of an adapter is configured to connect to a proximal end of a fluidic lumen of a multi-lumen catheter. In some embodiments, the stylet port of the adapter is configured to connect to a proximal end of a guide lumen of a multi-lumen catheter.

In some embodiments, a connector port of an adapter is configured to connect to a fluid line (e.g., medical tubing comprising a fluid). For example, FIG. 6A shows a computer automated drawing (CAD) of an exemplary adapter 4000 comprising a connector port 4100, fluid port 4200, and stylet port 4300. In some embodiments, connector port 4100 is configured to be connected to a fluid line via a threaded interface 4400 (e.g., a luer lock). In some embodiments, the threaded interface 4400 includes one or more helical ridges or grooves disposed on an external surface of the adapter and the corresponding helical grooves or ridges are disposed on an internal surface of the fluid line. The threads can be configured such that relative rotation between the connector port and the fluid line causes each to advance axially with respect to one another, thereby providing a secure mechanical engagement.

The threads may be continuous or discontinuous, single-start or multi-start, and may have a pitch, lead, or profile selected to achieve a desired engagement force, torque resistance, or ease of assembly. In some embodiments, the threads are compatible with a standard luer-lock type connection, although other thread geometries (e.g., square, buttress, V-form, or modified profiles) may also be employed. The threaded interface may be formed integrally with the components or may be provided as an insert, liner, or over mold.

In some embodiments, the threaded engagement can be supplemented by a taper, interference fit, or sealing feature (e.g., gasket, O-ring, compression surface) to enhance leak resistance and/or maintain sterility of the fluid path.

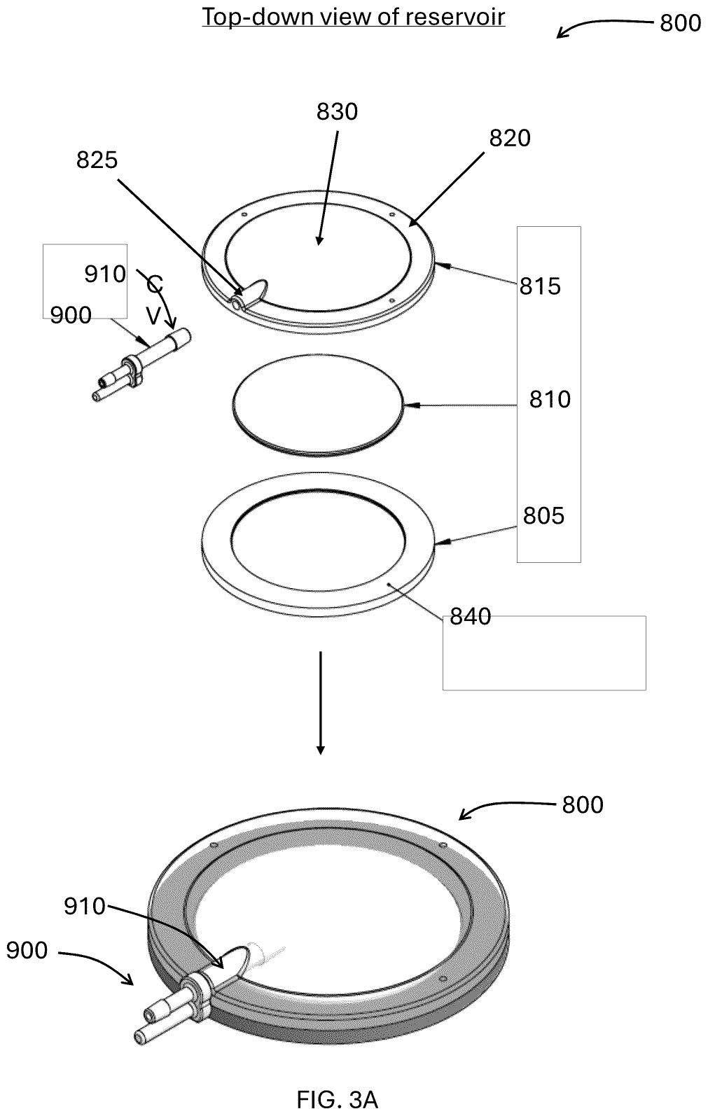

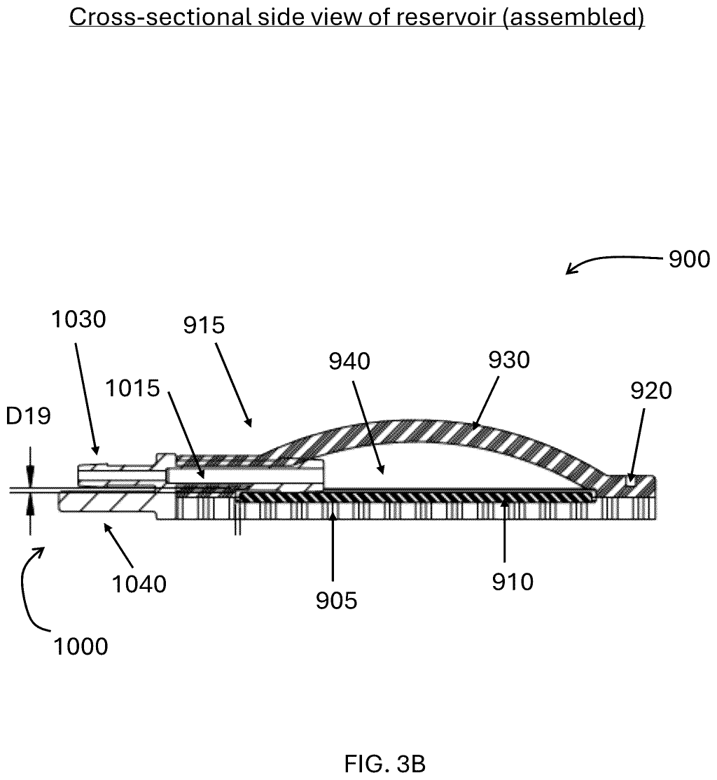

FIG. 6B shows a cross-sectional view of an exemplary adapter 5000 as contemplated herein. In some embodiments, adapter 5000 comprises connector port lumen 5100 and fluid port lumen 5200. In some embodiments, connector port lumen 5100 is in fluidic communication with fluid port lumen 5200 via fluid path 5300. In some embodiments, adapter 5000 further comprises a stylet port lumen, although this is not required in all embodiments. In some embodiments, the stylet port lumen is in fluidic communication with the fluid port lumen. In some embodiments, the stylet port lumen is in fluidic communication with the connector port lumen. In some embodiments, the connector port lumen, fluid port lumen, and stylet port lumen are in fluidic communication with one another. In some embodiments, style port 5400 does not comprise a lumen. FIG. 3A shows the assembly of exemplary reservoir 800, according to some embodiments of the present invention. In some embodiments, reservoir 800 comprises base 805, a rigid inner plate 810, and a top dome 815. In some embodiments, base 805 comprises an adhesive silicone 840. In some embodiments, top dome comprises an outer rim 820 comprising a fluid adapter port 825. In some embodiments, the fluid adapter port 825 is configured to receive a connector port 910 of an exemplary adapter 900. In some embodiments, the top dome further comprises an inner membrane 830. In some embodiments, inner membrane 830 is a self-healing membrane. As used herein, the term “self-healing membrane” refers to any membrane that does not leak following puncture with a needle (e.g., puncture with a 10 gauge (G) needle, 11 G, 12 G, 13 G, 14 G, 15 G, 16 G, 17 G, 18 G, 19 G, 20 G, 21 G, 22 G, 22 sG, 23 G, 23 sG, 24 G, 25 G, 25sG, 26 G, 26sG, 27 G, 28 G, 29 G, 30 G, 31 G, 32 G, 33 G, or a 34 G needle). FIG. 3A also shows a fully assembled reservoir 800 fitted with adapter 900.

FIG. 3B illustrates a cross-sectional side view of an exemplary reservoir 900, according to some embodiments. In some embodiments, a rigid inner plate 910 is enclosed between a top dome 915 and a base plate 905. In some embodiments, the inner membrane 930 is operably linked to the outer rim 920. In some embodiments, fluid cavity 940 is formed by placement of top dome 915 onto the base 905 and rigid inner plate 910. In some embodiments, the reservoir fluid cavity 940 is in fluidic communication with a connector port lumen 1015 of adapter 1000.

FIG. 3B also shows exemplary distance D19 between a tapered side of a fluid port 1040 and a first surface of a stylet port 1040 of exemplary adapter 1000. In some embodiments, distance D19 is between 0.1 mm and 0.3 mm. In some embodiments, the distance D19 between a tapered side of a fluid port and a first surface of a stylet port is greater than or equal to 0.1 mm, greater than or equal to 0.15 mm, greater than or equal to 0.2 mm, greater than or equal to 0.25 mm, or greater than or equal to 0.3 mm. In some embodiments, distance D19 between a tapered side of the fluid port and the first surface of the stylet port is less than or equal to 0.3 mm, less than or equal to 0.25 mm, less than or equal to 0.2 mm, less than or equal to 0.15 mm, or less than or equal to 0.10 mm. Combinations of the above recited ranges are also possible in some embodiments. For example, in some embodiments, the distance D19 between a tapered side of the fluid port and the first surface of the stylet port is of greater than or equal to 0.1 mm and less than or equal to 0.3 mm. In some embodiments, the distance D19 between a tapered side of the fluid port and the first surface of the stylet port is about 0.2 mm.

Any suitable materials known to the skilled artisan may be used to produce a reservoir as disclosed herein. Exemplary materials used to produce the inner membrane may include, but are not limited to, silicone, ethylene propylene diene rubber (EPDM), perfluoroelastomers (e.g., Isolast® Plus J9515, J9516, J9538 FFKM), fluoroelastomers (e.g., FKM), and acrylonitrile-butadiene rubber (e.g., NBR and HNBR). Exemplary materials used to produce the outer rim may include, but are not limited to, silicone, ethylene propylene diene rubber (EPDM), perfluoroelastomers (e.g., Isolast® Plus J9515, J9516, J9538 FFKM), fluoroelastomers (e.g., FKM), and acrylonitrile-butadiene rubber (e.g., NBR and HNBR). Exemplary materials used to produce the rigid inner plate may include, but are not limited to, biocompatible plastics such as, for example, polypropylene, polyethylene, PEEK, and the like. In some embodiments, the biocompatible plastics are rigid (e.g., they do not ready deform without an applied external pressure).

Additionally, any suitable method known to the skilled artisan may be used to operably link any two or more components of a reservoir as disclosed herein. For example, in some embodiments, two or more components (e.g., a base and top dome) are operably linked using silicone adhesive.

In some embodiments, a device or system comprise one or more multi-lumen catheters, adapters, ports, and/or reservoirs, as described above. For example, in some embodiments, the device comprises the multi-lumen catheter and an adapter. In other embodiments, a system comprises the multi-lumen catheter, an adapter, and a reservoir. In some embodiments, the one or more multi-lumen catheters, adapters, ports, and/or reservoirs comprise a plurality of components comprising one or more structures (e.g., catheters comprising guide lumen, fluid lumen, adhesive plug, tube, graduation markings, and central elongate passageways; adapters comprising connector ports, fluid ports, and stylet ports; reservoirs comprising fluid adapter port, inner membrane, outer rim, top dome, rigid inner plate, and base). When one or more components are present in a device or system, the amount of each type of structure present in the component (e.g., outer cross-sectional dimension D1, inner cross-sectional dimension D2, inner cross-sectional dimension D3, separation dimensions D4 and D5, length dimension D6, outer cross-sectional dimension D7, an inner cross-sectional dimension D8, length of dimension D9, inner diameters D10 and D11, wall thickness D12, diameters D13 and D14, wall thickness D15, distance D16, length D17, etc.) may independently be in one or more of the above-referenced ranges and/or all of the structures together may be present in an amount in one or more of the above-referenced ranges. When two or more components (e.g., a multi-lumen catheter, an adapter, and/or a reservoir) comprising the plurality of structures (e.g., outer cross-sectional dimension D1, inner cross-sectional dimension D2, inner cross-sectional dimension D3, separation dimensions D4 and D5, length dimension D6, outer cross-sectional dimension D7, an inner cross-sectional dimension D8, length of dimension D9, inner diameters D10 and D11, wall thickness D12, diameters D13 and D14, wall thickness D15, distance D16, length D17, etc.) are present, the preceding sentence may be independently true for each such additional component (e.g., a first multi-lumen catheter, second multi-lumen catheter, third multi-lumen catheter, etc.).

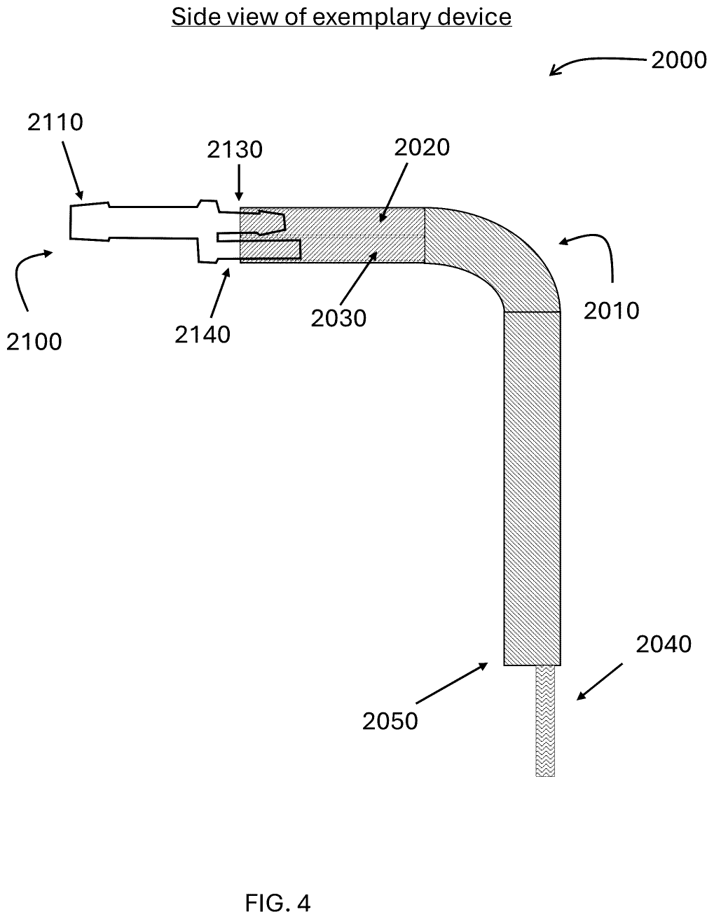

FIG. 4 shows an exemplary device 2000, according to some embodiments of the present invention. Device 2000, according to some embodiments, comprises a size-adjustable multi-lumen catheter 2010 comprising a fluidic lumen 2020 and a guide lumen 2030. Any of the multi-lumen catheters disclosed herein may be used in the devices of the present disclosure. In some embodiments, the multi-lumen catheter 2010 further comprises a tube 2040 comprising a central elongate passageway. In some embodiments, the tube 2040 is sized and configured to be reversibly positioned within at least a portion of the fluidic lumen 2020 such that the tube extends beyond a distal end 2050 of the multi-lumen catheter 2010. Additionally, in some embodiments, the device further comprises an adapter 2100 comprising a fluid port 2130 configured to be positioned within a proximal end of the fluidic lumen 2020 of the multi-lumen catheter 2010 and a stylet port 2140 configured to be positioned within a proximal end of the guide lumen 2030 of the multi-lumen catheter.

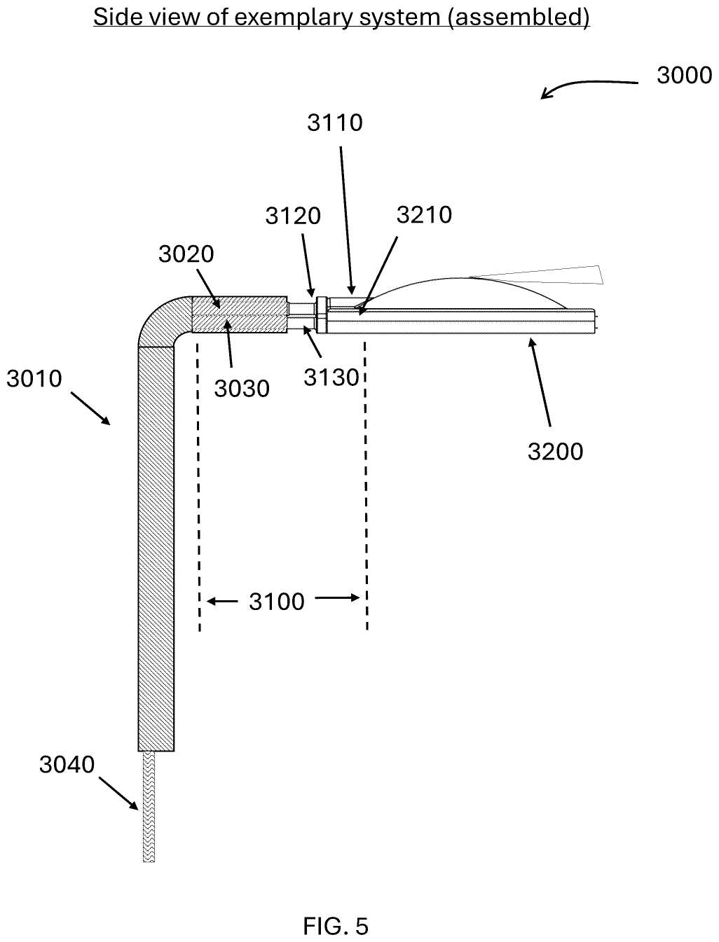

FIG. 5 illustrates exemplary system 3000 as contemplated herein. In some embodiments, system 3000 comprises a multi-lumen catheter 3010 comprising a fluidic lumen 3020, a guide lumen 3030 adjacent the fluidic lumen 3020, and a tube 3040, the tube comprising a central elongate passageway. In some embodiments, the tube 3040 is sized and configured to be reversibly positioned within at least a portion of the fluidic lumen 3020 such that the tube 3040 extends beyond a distal end of the multi-lumen catheter 3010. In some embodiments, the system further comprises a fitted adapter 3100 and a reservoir 3200 comprising a fluid adapter port 3210. In some embodiments, the fluid adapter port 3210 comprises one or more openings, each opening corresponding to one or more features of the fitted adapter 3100 such that the fitted adapter 3100 connects to the fluid adapter port 3210 thereby positioning the reservoir 3200 in fluidic communication with the fluidic lumen 3020 of the multi-lumen catheter 3010.

For example, in some embodiments, a fitted adapter 3100 comprises a connector port 3110 configured to be positioned within the fluid adapter port 3210. In some embodiments, the connector port 3110 of the fitted adapter 3100 is in fluidic communication with a fluid port 3120 of the fitted adapter 3100, which is in turn, configured to be placed within a fluidic lumen 3020 of a multi-lumen catheter 3010.

In some embodiments, a connector port of an adapter is connected to a fluid adapter port. In some embodiments, a connector port may be joined, coupled, or otherwise connected to the fluid adapter port using any suitable attachment technique known in the art. Such attachment may be permanent or releasable, and may be achieved, for example, by threaded engagement, frictional fit, snap-fit, press-fit, bayonet coupling, magnetic coupling, adhesive bonding, ultrasonic bonding, thermal fusion, or combinations thereof.

In some embodiments, the connection may further include a sealing feature, such as a tapered surface, gasket, O-ring, compression seal, or interference fit, to provide a fluid-tight and/or air-tight interface. In some embodiments, the connection is primarily structural and does not require sealing.

Unless otherwise specified, references to “connection,” “coupling,” or “attachment” herein are intended to broadly encompass direct and indirect engagements between a first component (e.g., connector port) and the second component (e.g., fluid adapter port), including engagements that permit disassembly and reassembly, engagements that are fixed and non-releasable, and engagements that may rely on mechanical, chemical, magnetic, or other securing forces.

In other embodiments, the system is configured to administer a fluid to a subject. In some embodiments, the system comprises a catheter (e.g., a multi-lumen catheter) comprising a first lumen (e.g., a fluidic lumen) and a second lumen (e.g., a stylet lumen), the first lumen (e.g., fluidic lumen) having a first diameter and the second lumen (e.g., stylet lumen) having a second diameter. In some embodiments, the system further comprises a fluidic reservoir comprising a fluid adapter port comprising a first opening having a first cross-sectional shape. In some embodiments, the system further comprises a fitted adapter comprising a first plug (e.g., connector port), a second plug (e.g., a fluid port), and a third plug (e.g., a stylet port). In some embodiments, the first plug (e.g., connector port) has a second cross-sectional shape corresponding to the first cross-sectional shape (e.g., cross-sectional shape of fluid adapter port). In some embodiments, the second plug (e.g., fluid port) is sized and adapted to operably connect to the first lumen (e.g., fluidic lumen of the multi-catheter port), thus placing the first plug (e.g., connector port) and second plug (e.g., fluid port) in fluidic communication. In some embodiments, the third plug (e.g., stylet port) is sized and adapted to operably connect to a second lumen (e.g., guide lumen of the multi-catheter port). In some embodiments, the third plug (e.g., stylet port) is not in fluidic communication with the first plug (e.g., connector port) or the second plug (e.g., fluid port). In some embodiments, the third plug (e.g., stylet port) is in fluidic communication with the first plug (e.g., connector port) and/or the second plug (e.g., fluid port). In some embodiments, the second plug (e.g., fluid port) comprises a first surface and the third plug (e.g., stylet port) comprises a second surface. In some embodiments, a shape of a planar surface of the first surface is different than a shape of a planar surface of the second surface.

It will be appreciated that any of the embodiments described herein are provided for illustrative purposes only and are not intended to be limiting in any way. For example, the connector port of an adapter may be connected to a fluid reservoir port of a reservoir (e.g., implanted within the scalp) or to a separate fluid line (e.g., external of the scalp) using any suitable connection known to one of ordinary skill. Accordingly, in some embodiments, a connector port is connected to a “second port”through any suitable engagement mechanism. Such connections may include, without limitation, a male-female interface, wherein a projecting portion of one end is received within a corresponding receptacle of the other end. In some embodiments, the connection may be achieved through threaded engagement, such as straight threads, tapered threads, or other threaded unions.

In some embodiments, the connector port may be connected to a second port using a quick-connect feature, such as a latch, snap-fit, bayonet, or push-to-connect mechanism, which allows for rapid assembly and disassembly. Still further, in some embodiments, the connection may be provided by a non-threaded, non-Luer-type fitting, including a friction fit, interference fit, pressure fit, compression fit, or barbed fitting, each of which may provide retention and, in some cases, a fluid-tight seal.

In some embodiments, the connection may be configured to be either permanent or releasable, and may optionally include additional sealing elements such as O-rings, gaskets, adhesives, or compression members to ensure integrity under fluid pressure, vacuum, or other operational conditions.

Further, in some embodiments, the multi-lumen catheters, adapters, reservoirs, devices, and/or systems disclosed herein comprise one or more valves to regulate, restrict, or permit fluid communication between various components of the devices and systems disclosed herein (e.g., between a connector port, fluid port, and stylet port of an adapter). The valve may take any suitable form, including but not limited to: check valves, one-way valves, relief valves, shut-off valves, pinch valves, ball valves, needle valves, diaphragm valves, poppet valves, and rotary valves. In some embodiments, the valve may be manually actuated, for instance by rotation, depression, or sliding of a control element. In other embodiments, the valve may be automatically actuated, for example by spring bias, fluid pressure differential, solenoid, or other electromechanical actuator. In some embodiments, the valve may be integral to a housing or conduit (e.g., anchor), while in other forms it may be a separate component coupled in-line between a first and second fluid path.

The sealing of the valve may be accomplished using any suitable structure, such as O-rings, gaskets, diaphragms, elastomeric seats, or interference-fit surfaces, and may be selected based on the desired fluid pressure rating, chemical compatibility, or sterilization requirements.

In some embodiments, the valve may is adapted for use with liquid, gas, or multiphase fluids. In some embodiments, the valve is designed to withstand positive pressure, vacuum, or both. In some embodiments, the valve is constructed, for example, using metals, polymers, ceramics, composites, or combinations thereof.

Additional aspects of the disclosure relate to methods of inserting the described multi-lumen catheters, adapters, ports and/or systems into a target tissue of a subject (e.g., brain tissue). In some embodiments, the methods relate to inserting a catheter in a tissue of a subject (e.g., tumor tissue). In some embodiments, the methods comprise loading a stylet into a guide lumen at a proximal end of a multi-lumen catheter. The multi-lumen catheter, according to some embodiments, further comprises a fluidic lumen and a tube. The tube, in some embodiments, comprises a central elongate passageway and is configured to be positioned within at least a portion of the fluidic lumen and to extend beyond a distal end of the multi-lumen catheter. In some embodiments, the methods further comprise using the stylet to insert and advance the catheter to a target location within the tissue of the subject. In some embodiments, the methods further comprise removing the stylet from the guide lumen of the multi-lumen catheter and cutting the multi-lumen catheter to a final length. In some embodiments, the methods comprise connecting an adapter, comprising a connector port, a fluid port, and a stylet port, to the multi-lumen catheter. In some embodiments, the fluid port of the adapter is connected to a proximal end of a fluidic lumen of the multi-lumen catheter. Additionally, in some embodiments, the stylet port of the adapter is connected to the proximal end of the guide lumen of the multi-lumen catheter. In some embodiments, the connector port is connected to a fluid source (e.g., a drug).

Those of skill in the art will understand that the above related methods can be used to insert a multi-lumen catheter into any suitable tissue known to the skilled artisan. Exemplary tissue include, but is not limited to, connective tissue (e.g., fat and other soft padding tissue, bone, and tendons), epithelial tissue (e.g., lining of GI tract organs and other hollow organs, skin surfaces, muscle tissue (e.g., cardiac muscle, smooth muscle, skeletal muscle), and nervous tissue (e.g., brain tissues, spinal cord tissue, and nerves). In some embodiments, the tissue is healthy tissue (e.g., not diseased tissue). In other embodiments, the tissue is a diseased tissue (e.g., tissue comprising cancer, inflammation, etc.).

In some embodiments, the methods comprise inserting the described multi-lumen catheters, adapters, ports, and/or systems into a target cavity of a subject (e.g., spinal cavity, brain ventricle, etc.). In some embodiments, the methods comprise loading a stylet into a guide lumen at a proximal end of the multi-lumen catheter. In some embodiments, the multi-lumen catheter further comprises a fluidic lumen and a tube. In some embodiments, the tube comprises a central elongate passageway configured to be positioned within at least a portion of the fluidic lumen. In some embodiments, the tube extends beyond a distal end of the multi-lumen catheter. In some embodiments, the methods further comprise using the stylet to insert and advance the multi-lumen catheter into the target cavity of the subject (e.g., spinal cavity, brain ventricle, etc.). In some embodiments, the methods further comprise removing the stylet from the guide lumen of the multi-lumen catheter and cutting the multi-lumen catheter to a final length. In some embodiments, the methods further comprise anchoring the multi-lumen catheter to the subject. Any suitable method known to the skilled artisan may be used to anchor the multi-lumen catheter to subject (e.g., anchoring to the skull, anchoring to the skin, etc.). In some cases, the multi-lumen catheter may be anchored using multiple techniques. In some embodiments, the methods further comprise connecting an adapter, comprising a connector port, a fluid port, and a stylet port, to the multi-lumen catheter. In some embodiments, the fluid port of the adapter is connected to a proximal end of a fluidic lumen of the multi-lumen catheter. Additionally, in some embodiments, the stylet port of the adapter is connected to the proximal end of the guide lumen of the multi-lumen catheter. In some embodiments, the connector port is subsequently connected to a fluid source (e.g., a drug). In some embodiments, at least a portion of the proximal end (e.g., the end interfacing with the fluid adapter) of the multi-lumen catheter is implanted transcutaneously (e.g., the adapter and at least a portion of the proximal end of the multi-lumen catheter is outside the skin).

Those of skill in the art will understand, based upon the teachings of this specification, that the above related methods can be used to insert a multi-lumen catheter into any suitable body cavity known to the skilled artisan. Exemplary body cavities include, but is not limited to cranial cavity, dorsal cavity, spinal cavity, pelvic cavity, abdominopelvic cavity, abdominal cavity, thoracic cavity, and/or ventral cavity. Other cavities in the body may also be accessed using the methods disclosed herein. For example, in some embodiments, the cavity is a brain ventricle or a vertebral cavity. In some embodiments, the methods disclosed herein can be used to insert the multi-lumen catheter into a canal within a body cavity. Exemplary canals within the cranial cavity include the alveolar canal, carotid canal, facial canal, greater palatine canal, incisive canal, infraorbital canal, mandibular canal, optic canal, palatovaginal canal, and the pterygoid canal. Exemplary canals in the abdominal cavity include the inguinal canal. Exemplary canals in the pelvic cavity include the anal canal, cervical canal, and the pudendal canal. In some embodiments, the canals may be in an upper or lower extremity. Exemplary canals in the upper extremities include the suprascapular canal, carpal canal, ulnar canal, and the radial canal; whereas exemplary canals in the lower extremities include the adductor canal, femoral canal, and the obturator canal.

In some embodiments, the methods relate to inserting a brain port comprising a reservoir in fluidic communication with a multi-lumen catheter in a brain of a subject, wherein the multi-lumen catheter comprises a fluidic lumen and a guide lumen and a tube comprising a central elongate passageway configured to be positioned within at least a portion of the fluidic lumen and to extend beyond at a distal end of the multi-lumen catheter. In some embodiments, the methods further comprise inserting a connector port of an adapter into a fluid adapter port of the reservoir. In some embodiments, the adapter further comprises a fluid port and a stylet port (e.g., at an opposing end, relative to the connector port, of the adapter). In some embodiments, the methods comprise inserting the reservoir in a subgaleal space of the subject. In some embodiments, the methods comprise loading a stylet at a proximal end of the guide lumen of the multi-lumen catheter and advancing the distal end of the multi-lumen catheter through the brain until a target location is reached. In some embodiments, the methods further comprise removing the stylet from the proximal end of the guide lumen of the multi-lumen catheter and cutting the multi-lumen catheter to a final length (e.g., using scissors or surgical blade). In some embodiments, the methods further comprise connecting the stylet port of the adapter to the proximal end of the guide lumen of the multi-lumen catheter and the fluid port of the adapter to the proximal end of the fluidic lumen of the multi-lumen catheter.

The following description outlines an exemplary method for implanting a brain port into the brain of a subject. More specifically, the exemplary method is an illustrative surgical approach using image guided navigation and a navigated stylet. Those of skill in the art will understand, however, that such aids are optional and are not required to perform the methods disclosed herein.

In some embodiments, inserting a brain port begins with selecting a target location for a multi-lumen catheter tip (e.g., the tip of the tube in FIG. 1A), for example, using an image guidance system; optionally, an entry point is also pre-selected so that the trajectory can be evaluated pre-operatively. Those of skill in the art will understand however, that the entry point may be determined intraoperatively. In some embodiments, the methods further comprise positioning the subject in a desired orientation and registering the patients position to an image guidance system. In some embodiments, the methods further comprise using the systems navigation probe to identify the catheter entry point in the skull and marking this location. In some embodiments, the marked site is used to plan the incision, which needs to be large enough to allow placement of the reservoir in the subgaleal space. In some embodiments, the methods comprise sterilizing the marked site (e.g., shaving the region, draping the region, applying iodine solution to region). In some embodiments, the methods comprise making the incision in the scalp and creating a subgaleal pocket to receive the reservoir. In some embodiments, the methods further comprise creating a twist-drill burr hole (or alternatively a conventional burr hole) at the planned entry site.

In some embodiments, the methods further comprise inserting the adapter into the reservoir, filling the reservoir with saline and placing the saline filled reservoir into the subgaleal pocket. In some embodiments, the methods further comprise loading a stylet into a guide lumen of the multi-lumen catheter and loading saline into a fluid lumen of the multi-lumen catheter. In some embodiments, the methods further comprise using the image guided system configured to position a device holder such that it provides the correct trajectory form the planned entry point to the target location. In some embodiments, a drill guide is placed into the device holder and a twist drill burr hole is created in the skull of the subject. In some embodiments, the drill guide is removed following creation of the twist drill burr hole. In some embodiments, a catheter guide (e.g., a reducing tube) may be used guide the multi-lumen catheter to a predefined (and/or real-time) visualized stereotactic (and/or image guided) location in the target tissue.

In some embodiments, the methods further comprise perforating a dura of the subject. Any suitable method for perforating the dura known to the skilled artisan may used in the method as disclosed herein. Suitable methods include, but are not limited to, using a Kelly Probe (e.g., an insulated probe that has uninsulated ends and allows for use of electrocautery to perforate the dura) or a probe with a sharp tip.

In some embodiments, the methods further comprise advancing the multi-lumen catheter to the target location. Any suitable method for advancing the multi-lumen catheter to the target location known to the skilled artisan may be used in the methods disclosed herein. For example, in some embodiments, a navigated stylet is used to advance the multi-lumen catheter to the target location. This is accomplished, for example, by entering an offset into an image guidance system to account for the length of the tube extending beyond the distal end of fluid lumen of the multi-lumen catheter. Using this approach, the image guidance system advances the catheter in real time until the tip reaches the target location (e.g., with the offset accounted for). Alternatively, the multi-lumen catheter may be advanced to the target location using a non-navigated stylet, according to some embodiments. Using this approach requires (1) determining the distance between the probe holder and the target location using an image guidance system, (2) determining any offset relating to the distance from the bottom of the probe holder (e.g., which is what the image guidance system may use as its reference in calculating the distance to the target location), (3) marking the graduation label on the multi-lumen catheter corresponding to the total distance to the target location (e.g., sum of steps (1) and (2)), (3) manually advancing the multi-lumen catheter through the probe holder until the marked graduation label is at the top of the holder, (4) removing the stylet from the stylet lumen of the multi-lumen catheter, (5) trimming the multi-lumen catheter to the appropriate length to allow for connection to an adapter which sits within a fluid adapter port of the reservoir, which sits within the subgaleal pocket, (6) connecting the fluid lumen of the multi-lumen catheter to the fluid port of the adapter and the style lumen of the multi-lumen catheter to the stylet port of the adapter, (7) securing the multi-lumen catheter to the adapter using a silk tie (e.g., securing the connection between fluid port of the adapter to the proximal end of the fluidic lumen of the multi-lumen catheter using a silk tie and securing the connection between the stylet port of the adapter and the proximal end of the guide lumen of the multi-lumen catheter using a silk tie), and (8) closing the incision in the scalp using known techniques in the art.

Additional methods are related to accessing a reservoir of a brain port, for example, after implantation within a skull of a patient. In some embodiments, the methods comprise preparing and sterilizing the scalp above where the port is located. In some embodiments, the methods comprise connecting a needle to an infusion line (e.g., a 25G needle or less comprising a Luer lock) primed with saline and/or a drug of interest. In some embodiments, the methods comprise inserting the needle into a fluid cavity of the reservoir (e.g., through the inner membrane) and infusing the saline and/or drug of interest into the fluid cavity of the reservoir. In some embodiments, the methods further comprise removing the needle from the reservoir and placing a wound dressing over the injection site (e.g., to prevent infection). Those of skill in the art will appreciate that such methods may be useful, for example, for repeatedly delivering a chemotherapy drug, a radiotherapy drug, an immunotherapy drug, or a combination thereof, to a brain tumor.

In some embodiments, any of the methods disclosed herein may be performed using an image guided navigation system and a navigated stylet. In other embodiments, the methods disclosed herein may be performed using the image guided navigation system and a non-navigated stylet. When an image guided navigation system is used, the methods further comprise entering an offset into the image guidance system to account for the length of the tube extending beyond the distal end of the multi-lumen catheter.

In some embodiments, any of the methods disclosed herein further comprise filling a fluid cavity of a reservoir with saline (e.g., prior to placement within the subgaleal space). In some embodiments, the methods disclosed herein further comprise securing a connection between a stylet port of an adapter and a proximal end of a guide lumen of a multi-lumen catheter (e.g., using a silk tie). In some embodiments, the methods disclosed herein further comprise securing the connection between a fluid port of the adapter to the proximal end of the fluidic lumen of the multi-lumen catheter (e.g., using a silk tie).

In some embodiments, the methods relate to accessing a brain port as described above and using convention enhanced diffusion (CED) to deliver a drug to a reservoir of the brain port. Without wishing to be bound by any particular theory, CED is a method of drug delivery in which a drug (or combination of drugs) is delivered into a target location of a tissue (e.g., a brain tumor) using bulk flow rather than conventional diffusion. This is accomplished by inserting one or more catheters into a target location (e.g., brain tumor) and using fluid pressure (e.g., generated via infusion using an infusion pump) to push the drugs into the tissue at the target location. This method works, in essence, because bulk flow is limited only by Darcy's law, defined as v=−κ∇p, where v is the velocity, κ is the hydraulic conductivity of the molecule, and ∇p is the pressure gradient. Thus, using bulk flow allows a drug to be delivered further into a target tissue with higher pressure, resulting in lower concentrations of drug and less risk of drug-related toxicity.

In some embodiments, the methods comprise infusing (e.g., via an infusion pump) a fluid comprising a drug or a combination of drugs into a reservoir of a brain port using CED. In some embodiments, the fluid is infused into the brain port at a rate of between 0.1 and 10 microliters/min. In some embodiments, the fluid is infused into the reservoir of the brain port at a rate that is greater than or equal to 0.1 microliters/min, greater than or equal to 0.5 microliters/min, greater than or equal to 1 microliters/min, greater than or equal to 2 microliters/min, greater than or equal to 3 microliters/min, greater than or equal to 4 microliters/min, greater than or equal to 5 microliters/min, greater than or equal to 6 microliters/min, greater than or equal to 7 microliters/min, greater than or equal to 8 microliters/min, greater than or equal to 9 microliters/min, or greater than or equal to 10 microliters/min. In some embodiments, the fluid is infused into the reservoir of the brain port at a rate that is less than or equal to 10 microliters/min, less than or equal to 9 microliters/min, less than or equal to 8 microliters/min, less than or equal to 7 microliters/min, less than or equal to 6 microliters/min, less than or equal to 5 microliters/min, less than or equal to 4 microliters/min, less than or equal to 3 microliters/min, less than or equal to 2 microliters/min, less than or equal to 1 microliters/min, less than or equal to 0.5 microliters/min, or less than or equal to 0.1 microliters/min. Combinations of the above recited ranges are also possible in some embodiments. For example, in some embodiments, the fluid is infused into the reservoir of the brain port at a rate that is greater than or equal to 0.1 microliters/min and less than or equal to 10 microliters/min.

In some embodiments, one or more dimensions of a device disclosed herein (e.g., a diameter) may be expressed using the term “French (Fr).” Those of skill in the art will understand that 1 mm=3Fr. Thus, a recited range of 1 mm-10 mm would correspond to 3 Fr-30 Fr. According to some embodiments, the catheters, adapters, reservoirs, ports, and devices and systems comprising the same described herein are compatible with one or more therapeutic, diagnostic, and/or enhancement agents, such as drugs, nutrients, microorganisms, in vivo sensors, and tracers. In some embodiments, the active substance, is a therapeutic, nutraceutical, prophylactic or diagnostic agent. While much of the specification describes the use of therapeutic agents, other agents listed herein are also possible. For example, the fluids described herein may comprise an active substance.

Agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals, Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C.A. (ed.), Merck Publishing Group, 2005; and “Approved Drug Products with Therapeutic Equivalence and Evaluations,” published by the United States Food and Drug Administration (F.D. A.) (the “Orange Book”). Examples of drugs approved for human use are listed by the FDA under 21 C.F.R. § § 330.5, 331 through 361, and 440 through 460, incorporated herein by reference; drugs for veterinary use are listed by the FDA under 21 C.F.R. § § 500 through 589, incorporated herein by reference.

Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or nonsteroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

In certain embodiments, the therapeutic agent is cytotoxic chemotherapy, antimetabolic agent, antimitotic drug, targeted small molecule drug, therapeutic antibody, theragnostic agent, a virus, an oncolytic virus, a viral vector, an engineered viral vector, a cellular therapy, an engineered cellular therapy, stem cell therapy, and/or nucleic acid based therapeutics (e.g., antisense oligonucleotides, mRNA, siRNA, etc.).

### EXAMPLES

Example 1 relates to an exemplary catheter as contemplated herein. In some embodiments, the catheter, as shown in FIG. 1, comprises a tubular structure comprising a fluidic lumen and a guide lumen adjacent the fluidic lumen that both run the entire distance of the tubular structure. In some embodiments, the catheter further comprises a tube sized and configured to be reversibly positioned within at least a portion of the fluidic lumen such that it extends beyond a distal end of the multi-lumen catheter. In some embodiments, the tube is permanently positioned within at least a portion of the fluidic lumen such that it extends beyond a distal end of the multi-lumen catheter. Additionally, according to some embodiments, an adhesive plug is positioned at a distal end of the guide lumen (e.g., the distal end of the guide lumen is sealed). In other embodiments, still, the overall catheter length may be reduced (e.g., may be size-adjustable by cutting with scissors, a razor blade, etc.) as necessary, for example, to fit within a body cavity (e.g., within a brain cavity).
Example 2 relates to an exemplary adapter as contemplated herein. In some embodiments, the adapter, as shown in FIG. 2, comprises a connector port positioned at a first end of the adapter and a fluid port positioned at a second end of the adapter. In some embodiments, the fluid port is in fluidic communication with the connector port. Additionally, in some embodiments, the adapter comprises a stylet port, proximate the fluid port. In some embodiments, the stylet port further comprises a first surface and a second surface having different curvatures. In some embodiments, the stylet port extends beyond a distal portion of the fluid port.
Example 3 relates to an exemplary reservoir as contemplated herein. In some embodiments, the reservoir, as shown in FIG. 3, comprises a base, a rigid inner plate, and a top dome. The top dome, in some embodiments, further comprises a fluid adapter port that is configured to receive a fluid adapter, such as the adapter illustrated in FIG. 2. In some embodiments, a fluid cavity is formed by placement of the top dome onto the base and rigid inner plate. In some embodiment, the fluid adapter port is in fluidic communication with the reservoir fluid cavity.
Example 4 Relates to an exemplary device as contemplated herein. The device, according to some embodiments, is shown in FIG. 4 and comprises an adapter as shown in FIG. 2 and a catheter as described in FIG. 1. In some embodiments, the catheter is flexible and is configured to be positioned between −90 degrees and 90 degrees relative to a first axis that runs parallel to the fluid port of the fluid adapter. In some embodiments, the connector port is configured to be connected to a fluid line. In some embodiments, the distal end of the multi-lumen catheter is configured to be positioned within a tissue, cavity, or canal of a subject in need thereof.
Example 5 Relates to an exemplary system as contemplated herein. The system, according to some embodiments, is shown in FIG. 5 and comprises a reservoir as shown in FIG. 3, an adapter as shown in FIG. 2, and/or a catheter as described in FIG. 1. In some embodiments, the catheter is flexible and is configured to be positioned between −90 degrees and 90 degrees relative to a first axis that runs parallel to the fluid port of the fluid adapter. In some embodiments, the distal end of the multi-lumen catheter is configured to be positioned within a tissue, cavity, or canal of a subject in need thereof.

### Equivalents and Scope

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, gomboc, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system, comprising:
(i) a multi-lumen catheter, the catheter comprising:
a fluidic lumen;
a guide lumen adjacent the fluidic lumen; and
a tube, the tube comprising a central elongate passageway,
wherein the tube is sized and configured to be reversibly positioned within at least a portion of the fluidic lumen such that the tube extends beyond a distal end of the multi-lumen catheter;
(ii) a fitted adapter; and
(iii) a reservoir, the reservoir comprising a fluid adapter port,
wherein the fluid adapter port comprises one or more openings, each opening corresponding to one or more features of the fitted adapter such that the fitted adapter connects to the fluid adapter port thereby positioning the reservoir in fluidic communication with the fluidic lumen of the multi-lumen catheter.

2. A system configured for administration of a fluid to a subject, the system comprising:
a catheter comprising a first lumen and a second lumen, the first lumen having a first diameter and the second lumen having a second diameter;
a fluidic reservoir, the fluidic reservoir comprising a fluid adapter port having a first opening having a first cross-sectional shape;
a fitted adapter comprising a first plug, a second plug, and a third plug;
the first plug having a second cross-sectional shape corresponding to the first cross-sectional shape;
the second plug sized and adapted to operably connect to the first lumen, wherein the first plug and the second plug are in fluidic communication,
the third plug sized and adapted to operably connect to the second lumen, wherein the third plug is not in fluidic communication with the first plug, and
the second plug having a first surface, the third plug having a second surface, wherein a shape of a planar surface of the first surface is different than a shape of a planar surface of the second surface.

3. A device, comprising:
(i) a size-adjustable multi-lumen catheter comprising a fluidic lumen and a guide lumen, the size-adjustable catheter further comprising a tube, the tube comprising a central elongate passageway, wherein the tube is sized and configured to be reversibly positioned within at least a portion of the fluidic lumen such that the tube extends beyond a distal end of the multi-lumen catheter; and
(ii) an adapter comprising a fluid port configured to be positioned within a proximal end of the fluidic lumen of the catheter and a stylet port configured to be positioned within a proximal end of the guide lumen of the multi-lumen catheter.

4-17. (canceled)

18. The system as in claim 1, further comprising a plug, the plug sized and configured to be positioned within a distal portion of the guide lumen of the multi-lumen catheter.

19-27. (canceled)

28. The system as in claim 1, wherein the tube extends beyond a distal end of the multi-lumen catheter by between 5 mm and 20 mm.

29. (canceled)

30. The system as in claim 1, wherein the tube comprises fused silica.

31. The system as in claim 1, wherein the reservoir further comprises a rigid inner plate, a base plate, and/or a top dome.

32-33. (canceled)

34. The system as claim 31, wherein the rigid inner plate is enclosed between the top dome and the base plate.

35. The system as in claim 31, wherein the top dome comprises the fluid adapter port.

36. The system as in claim 1, wherein the fluid adapter port of the reservoir is configured to receive a connector port of the adapter.

37. The system as in claim 1, wherein the fitted adapter further comprises a connector port, a fluid port, and a stylet port.

38. The system as in claim 37, wherein a flat surface of the stylet port extends about 1+/−0.20 mm past the end of the fluid port.

39. The system as in claim 37, wherein one or more sides of the connector port and/or fluid port of the fluid-fitted adapter are tapered.

40. The system as in claim 37, wherein the connector port of the adapter has a larger inner diameter than the fluid port of the adapter.

41. The system as in claim 37, wherein the connector port of the adapter is configured to connect to the fluid adapter port of the reservoir.

42. The system as in claim 37, wherein the fluid port of the fitted adapter is configured to connect to a proximal end of the fluidic lumen of the multi-lumen catheter.

43. The system as in preceding claim 37, wherein the stylet port of the adapter is configured to connect to a proximal end of the guide lumen of the multi-lumen catheter.

44-48. (canceled)

49. The system as in claim 43, further comprising securing the connection between the stylet port of the fitted adapter and the proximal end of the guide lumen of the catheter using a silk tie.

50. The system as in claim 42, further comprising securing the connection between fluid port of the fitted adapter to the proximal end of the fluidic lumen of the catheter using a silk tie.

51. (canceled)

52. The system as in claim 37, wherein the connector port of the fitted adapter is configured to connect to a second port, wherein the second port is an external fluid line or a fluid adapter port of a reservoir.

53. The system as in any claim 37, wherein the connector port of the fitted adapter is connected to a second port by a connection selected from the group consisting of a male-female interface, a threaded engagement, a quick-connect interface, and a non-threaded interface.

54-58. (canceled)
